(19) United States
(12) Patent Application Publication
(10) Pub. No.: US 2022/0361774 A1
(43) Pub. Date: Nov. 17, 2022

(54) SYSTEMS AND METHODS FOR PERFORMING TRANS-ABDOMINAL FETAL OXIMETRY OR PULSE-OXIMETRY

(71) Applicant: Raydiant Oximetry, Inc., San Ramon, CA (US)

(72) Inventors: NEIL PADHARIA RAY, (US); MARK ANDREW ROSEN, Piedmont, CA (US); ADAM JACOBS, Hollis, NH (US); RODNEY P. CHIN, Rodeo, CA (US); DAVID BOAS, Boston, MA (US); JANA M. KAINERSTORFER, Pittsburgh, PA (US); RUSSELL DELONZOR, San Ramon, CA (US); DENISE ZARINS, Saratoga, CA (US)

(21) Appl. No.: 17/629,412

(22) PCT Filed: Jul. 24, 2020
(86) PCT No.: PCT/US2020/070312

§ 371 Date: Jan. 24, 2022

Related U.S. Application Data
(60) Provisional application No. 62/878,243, filed on Jul. 24, 2019.
(60) Provisional application No. 62/971,152, filed on Feb. 6, 2020.

(51) Int. Cl. A61B 5/1464; A61B 5/1455; A61B 5/1495

(52) U.S. Cl. CPC A61B 5/1464; A61B 5/14551; A61B 5/1495; A61B 2503/02; A61B 2560/0223

(57) ABSTRACT

Systems, devices, and methods for performing trans-abdominal fetal oximetry and/or trans-abdominal fetal pulse oximetry using physiological characteristics and/or a calibration factor may receive a physiological characteristic of a pregnant mammal and determine one or more potential impact(s) of the physiological characteristic on a behavior of an optical signal projected into the abdomen of the pregnant mammal Then a calibration factor for the optical signal responsively to the impact. The calibration factor may then be used to calibrate a fetal detected electronic signal so that a level of fetal hemoglobin oxygen saturation may be determined.

### RELATED APPLICATIONS

This application is an INTERNATIONAL PCT application of U.S. Provisional Patent Application Number 62/878,243 filed on Jul. 24, 2019 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING PHYSIOLOGICAL CHARACTERISITICS AND/OR A CALIBRATION FACTOR” and U.S. Provisional Patent Application Number 62/971,152 filed on Feb. 6, 2020 entitled “SYSTEMS, DEVICES, AND METHODS FOR PERFORMING FETAL OXIMETRY AND/OR FETAL PULSE OXIMETRY USING FETAL DEPTH AND/OR A MATERNAL HEMOGLOBIN OXYGEN SATURATION LEVEL” all of which are hereby incorporated, in their entireties, herein.

### FIELD OF INVENTION

The present invention is in the field of medical devices and, more particularly, in the field of trans-abdominal fetal oximetry and trans-abdominal fetal pulse oximetry.

### BACKGROUND

Oximetry is a method for determining the oxygen saturation of hemoglobin in a mammal's blood. Typically, 90% (or higher) of an adult human's hemoglobin is saturated with (i.e., bound to) oxygen while only 30-60% of a fetus's blood is saturated with oxygen. Pulse oximetry is a type of oximetry that uses changes in blood volume through a heartbeat cycle to internally calibrate hemoglobin oxygen saturation measurements of the arterial blood.

Current methods of monitoring fetal health, such as monitoring fetal heart rate, are inefficient at determining levels of fetal distress and, at times, provide false positive results indicating fetal distress that may result in the unnecessary performance of a Cesarean delivery.

### SUMMARY

Systems, devices, and methods for performing trans-abdominal fetal oximetry and/or trans-abdominal fetal pulse oximetry using physiological characteristics and/or a calibration factor are herein disclosed. In some embodiments, a physiological characteristic of a pregnant mammal may be received by, for example, a computer or processor. An impact of the physiological characteristic on a behavior of an optical signal projected into the abdomen of the pregnant mammal may then be determined. Exemplary impacts include absorption and scattering of the optical signal. A calibration factor for the optical signal may then be determined responsively to the determined impact of the physiological characteristic. In some cases, determining a calibration factor may include querying a database using the physiological characteristic for a corresponding calibration factor. The determined calibration factor may then be stored in a database.

On some occasions, the processor may further receive a composite detected electronic signal from a detector communicatively coupled to the processor. The composite electronic signal may correspond to an optical signal emitted from the pregnant mammal's abdomen and a fetus contained therein that has been detected by the detector and converted into the composite detected electronic signal. The emitted optical signal may be a portion of light projected into the pregnant mammal's abdomen and onto the fetus contained therein. A fetal signal may then be generated by isolating a portion of the composite detected electronic signal that corresponds to light that was incident upon the fetus. Isolation of the fetal signal from a composite (maternal and fetal signal) may be accomplished a number of ways including, but not limited to, filtering via, for example, bandpass or Kalman filters, amplification, and/or processing using one or more input signals such as fetal heart rate, maternal heart rate, maternal pulse oxygenation values, and/or maternal respiratory values to remove a portion of the composite signal contributed by the pregnant mammal and/or amplify a portion of the composite signal contributed by the fetus. Some of these techniques may also be used remove noise (e.g., ambient light, harmonics, etc.) from the composite and/or fetal signal. The calibration factor may then be applied to the fetal signal to generate a calibrated fetal signal and the calibrated fetal signal may be processed to determine a fetal hemoglobin oxygen saturation level for the fetus. The fetal hemoglobin oxygen saturation level may then be communicated to a user such as a doctor, midwife, or nurse.

In some embodiments, an indication of whether the fetal signal corresponds to pre-ductal or post-ductal blood may be received by the processor. Often times, this indication is input by a clinician based on a location of the detector detecting the composite signal on the pregnant mammal's abdomen that corresponds to a location on the fetus (e.g., head, thorax, or limb) from which the composite signal is generated. This indication may later be provided or displayed to a user along with the fetal hemoglobin oxygen saturation level so that the user may determine whether the fetal hemoglobin oxygen saturation level is dangerously low for the fetus.

In some embodiments, a maternal detected electronic signal may be received from a detector communicatively coupled to the processor. The maternal detected electronic signal may correspond to an optical signal emitted from the pregnant mammal's abdomen (that has not traveled deep enough into the abdomen to reach to the fetus) that has been detected by the detector and converted into the maternal detected electronic signal. In some embodiments, the maternal detected electronic signal may be a short separation signal that only passes through maternal tissue. The emitted optical signal may be a portion of light projected, by a light source, into the pregnant mammal's abdomen. Then, the maternal detected electronic signal may be analyzed to determine the physiological characteristic of the pregnant mammal. The determined physiological characteristic and/or the calibration factor for the pregnant mammal may be stored in a database.

The received physiological characteristic may be intrinsic or extrinsic and may be, for example, the pregnant mammal's age, the pregnant mammal's weight, and the pregnant mammal's body mass index. At times, the physiological characteristic is received from a clinician based on his or her observations, an ultra-sound device, a Doppler device, an image of the pregnant mammal's abdomen, a Fitzpatrick scale reading, manually-operated calipers, a blood measurement device, an oximeter, a pulse oximeter, and/or a scale.

In one embodiment, the received physiological characteristic is a skin color, or melanin concentration, of the pregnant mammal and the determination of the impact of the physiological characteristic on the behavior of the optical signal may include determining how much of the optical signal is absorbed by the pregnant mammal's melanin/skin color.

Additionally, or alternatively, the received physiological characteristic may be a thickness of a muscle layer in the pregnant mammal's abdomen. When this is the case, the determination of the impact of the physiological characteristic on the behavior of the optical signal may include determining how much of the optical signal is absorbed by the muscle layer in the pregnant mammal's abdomen.

Additionally, or alternatively the received physiological characteristic is a thickness of an adipose layer in the pregnant mammal's abdomen, further wherein the determination of the impact of the physiological characteristic on the behavior of the optical signal includes determining how much of the optical signal is scattered by the adipose layer in the pregnant mammal's abdomen.

Additionally, or alternatively, the received physiological characteristic may be a body mass index for the pregnant mammal and the determination of the impact of the physiological characteristic on the behavior of the optical signal may include determining how much of the optical signal is scattered or absorbed by the pregnant mammal's abdomen due to her body mass index.

Additionally, or alternatively, the received physiological characteristic may be a thickness of the pregnant mammal's abdomen (also referred to herein as fetal depth). In this case, the determination of the impact of the physiological characteristic on the behavior of the optical signal may include determining how much of the optical signal is absorbed by the pregnant mammal's abdomen/abdominal tissue.

Additionally, or alternatively, the received physiological characteristic may be a thickness of the pregnant mammal's abdomen and the determination of the impact of the physiological characteristic on the behavior of the optical signal may include determining how much of the optical signal is scattered by the pregnant mammal's abdomen.

Additionally, or alternatively, the received physiological characteristic may include a hemoglobin concentration of the pregnant mammal's blood. In these situations, the determination of the impact of the physiological characteristic on the behavior of the optical signal may include determining how much of the optical signal is absorbed by the pregnant mammal's hemoglobin.

Additionally, or alternatively, the received physiological characteristic may be a hemoglobin oxygen saturation of the pregnant mammal's blood the determination of the impact of the physiological characteristic on the behavior of the optical signal may include determining how much of the optical signal is absorbed by the pregnant mammal's oxygenated and/or deoxygenated hemoglobin.

In another embodiment, a maternal detected electronic signal may be received from a detector communicatively coupled to a processor, the maternal detected electronic signal may correspond to an optical signal emitted from the pregnant mammal's abdomen that has been detected by the detector and converted into the maternal detected electronic signal. The emitted optical signal may be a portion of light projected (by a light source) into the pregnant mammal's abdomen. The maternal detected electronic signal may then be analyzed to determine a physiological characteristic of the pregnant mammal. A calibration factor for the optical signal emanating from the pregnant mammal may then be determined responsively to the analysis. In some embodiments, the physiological characteristic of the pregnant mammal may be associated with the calibration factor and this association may be stored in a database.

In some instances, a composite detected electronic signal may be received from a detector communicatively coupled to the processor. The composite detected electronic signal may correspond to an optical signal emitted from the pregnant mammal's abdomen and a fetus contained therein that has been detected by the detector and converted into the composite detected electronic signal. The emitted optical signal may be a portion of light projected, by a light source, into the pregnant mammal's abdomen and onto the fetus contained therein. A fetal signal may then be generated by isolating a portion of the composite detected electronic signal that corresponds to light that was incident upon the fetus. A calibrated fetal signal may be generated by applying the calibration factor to the fetal signal. Then, a fetal hemoglobin oxygen saturation level may be determined using the calibrated fetal signal and the fetal hemoglobin oxygen saturation may be communicated to a user via, for example, displaying the fetal hemoglobin oxygen saturation on a display device.

In some embodiments, determining the calibration factor for the optical signal responsively to the impact includes querying a database for a calibration factor that corresponds to the physiological characteristic and receiving the queried-for calibration factor from the database.

In some instances, an indication of whether the fetal signal corresponds to pre-ductal or post-ductal blood may be received from, for example, a clinician or doctor and this indication may be provided along with the fetal hemoglobin oxygen saturation level to the user.

On some occasions, a maternal detected electronic signal may be received from a detector communicatively coupled to the processor. The maternal detected electronic signal may correspond to an optical signal emitted from the pregnant mammal's abdomen that has been detected by the detector and converted into the maternal detected electronic signal that has not passed through, or been incident upon, the fetus. Thus, it is an optical signal that only passes through the maternal abdomen and does not penetrate far enough into the abdomen to be incident on the fetus. The maternal detected electronic signal may then be analyzed and/or processed, and the physiological characteristic of the pregnant mammal may be determined responsively to the analysis.

In some cases, the determined physiological characteristic is a skin color of the pregnant mammal and the calibration factor may pertain to how much of the optical signal is absorbed by the pregnant mammal's skin color. Additionally, or alternatively, the determined physiological characteristic may be a thickness of a muscle layer in the pregnant mammal's abdomen and the calibration factor may pertain to how much of the optical signal is absorbed by the muscle layer in the pregnant mammal's abdomen.

Additionally, or alternatively, the determined physiological characteristic may be a thickness of an adipose layer in the pregnant mammal's abdomen and the calibration factor may pertain to how much of the optical signal may be scattered by the adipose layer in the pregnant mammal's abdomen. Additionally, or alternatively, the determined physiological characteristic may be a thickness of the pregnant mammal's abdomen and the calibration factor may pertain to how much of the optical signal may be absorbed by the pregnant mammal's by the pregnant mammal's abdomen. Additionally, or alternatively, the determined physiological characteristic may be a thickness of the pregnant mammal's abdomen and the calibration factor may pertain to how much of the optical signal is scattered by the pregnant mammal's abdomen. Additionally, or alternatively, the determined physiological characteristic may be a hemoglobin concentration of the pregnant mammal's blood and the calibration factor may pertain to how much of the optical signal is absorbed by the pregnant mammal's hemoglobin. Additionally, or alternatively, the determined physiological characteristic may be a hemoglobin oxygen saturation of the pregnant mammal's blood and the calibration factor may pertain to how much of the optical signal is absorbed by the pregnant mammal's oxygenated and deoxygenated hemoglobin.

### BRIEF DESCRIPTION OF THE FIGURES

FIG. 1A is a block diagram illustrating an exemplary system for determining a level of oxygen saturation for fetal hemoglobin and/or whether meconium is present in the amniotic fluid of a pregnant mammal, consistent with some embodiments of the present invention;

FIG. 1B is a block diagram of an exemplary processor-based system that may store data and/or execute instructions for the processes disclosed herein, consistent with some embodiments of the present invention;

FIG. 2A is a block diagram illustrating an exemplary fetal probe, consistent with some embodiments of the present invention;

FIG. 2B is a block diagram illustrating another exemplary fetal probe, consistent with some embodiments of the present invention;

FIG. 3A provides an illustration of exemplary dimensions for layers of tissue within two different maternal abdomens with their respective fetuses, consistent with some embodiments of the present invention;

FIG. 3B provides an illustration of exemplary dimensions for layers of tissue within two different maternal abdomens with their respective fetuses, consistent with some embodiments of the present invention;

FIG. 3C provides a midsagittal plane view of pregnant mammal's abdomen with fetal hemoglobin probe positioned thereon, consistent with some embodiments of the present invention;

FIG. 4A illustrates an exemplary fetal hemoglobin probe in contact with a pregnant mammal's abdomen showing the different layers of maternal abdominal tissue, consistent with some embodiments of the present invention;

FIG. 4B illustrates another exemplary fetal hemoglobin probe in contact with a pregnant mammal's abdomen, consistent with some embodiments of the present invention;

FIG. 4C illustrates an exemplary fetal probe configured to detect two short separation signals and one long separation signal in contact with a pregnant mammal's abdomen where the layers of the maternal abdomen are depicted as a single layer, consistent with some embodiments of the present invention;

FIG. 4D illustrates an exemplary fetal probe configured to detect two short separation signals and one long separation signal in contact with a pregnant mammal's abdomen where some of the layers of the maternal abdomen are shown, consistent with some embodiments of the present invention;

FIG. 5 provides a flowchart illustrating a process for determining a fetal hemoglobin oxygen saturation level, consistent with some embodiments of the present invention;

FIG. 6 provides a flowchart illustrating a process for determining a physiological characteristic of a pregnant mammal using a received optical signal consistent with some embodiments of the present invention;

FIG. 7A is a flowchart illustrating an exemplary process for determining a fetal depth and/or a fetal hemoglobin oxygen saturation level, in accordance with some embodiments of the present invention;

FIG. 7B provides a flowchart illustrating an exemplary process for determining a fetal depth, in accordance with some embodiments of the present invention;

FIG. 7C provides a graph showing a scatter plot of a change in percent transmission of light for 1^{st}-N^{th} fetal signals as a function of source/detector distance, in accordance with some embodiments of the present invention;

FIG. 7D provides a graph showing a scatter plot of a change in percent transmission of light for 1^{st}-N^{th} maternal signals as a function of source/detector distance, in accordance with some embodiments of the present invention;

FIG. 8 is a flowchart illustrating an exemplary process for determining a fetal depth and/or a fetal hemoglobin oxygen saturation level, in accordance with some embodiments of the present invention;

FIG. 9 is a flowchart illustrating an exemplary process for determining a fetal depth and/or a fetal hemoglobin oxygen saturation level, in accordance with some embodiments of the present invention;

FIG. 10 provides a flowchart illustrating a process for determining a fetal hemoglobin oxygenation saturation level using physiological characteristics of the pregnant mammal determined using one or more maternal detected electronic signal(s), in accordance with some embodiments of the present invention;

FIG. 11 provides a flowchart illustrating a process 1100 for determining an influence of a physiological characteristic on the behavior of light traversing through the abdomen of a pregnant mammal and/or her fetus, in accordance with some embodiments of the present invention;

FIG. 12 is a flowchart illustrating an exemplary process for determining a fetal hemoglobin oxygen saturation level using a maternal hemoglobin oxygen saturation level and/or a fetal depth, in accordance with some embodiments of the present invention;

FIG. 13 provides a flowchart illustrating a process for determining a fetal hemoglobin oxygenation saturation level using calibration factor and/or physiological characteristic of the pregnant mammal and/or fetus, consistent with some embodiments of the present invention;

FIG. 14 provides a flowchart illustrating a process for determining a fetal hemoglobin oxygenation saturation level, consistent with some embodiments of the present invention;

FIG. 15A provides a flowchart illustrating a first part of a process for determining a composite fetal hemoglobin oxygenation saturation level, consistent with some embodiments of the present invention; and

FIG. 15B provides a flowchart illustrating a second part of a process for determining a composite fetal hemoglobin oxygenation saturation level, consistent with some embodiments of the present invention.

### WRITTEN DESCRIPTION

Behavior of light projected into the abdomen of a pregnant mammal may be impacted (e.g., absorbed and/or scattered) by the abdominal tissue of the pregnant mammal. This may impact how much light incident on the maternal abdomen is incident on a fetus within the pregnant mammal's abdomen and/or a clarity of a signal received from the maternal abdomen and/or a signal that was incident on the fetus. Knowing how much light is incident on a fetus may be important for various reasons. For example, a value for the intensity of light incident on a fetus and/or a percent transmission of light through the pregnant mammal's abdomen may be used to calculate fetal hemoglobin oxygen saturation using the oximetry calculations and/or the Beer-Lambert Law. Also, understanding the behavior (absorption and/or scattering, which may also be referred to herein as absorption coefficients, or (μₐ(λ)), and/or scattering coefficients (μₛ(λ)) for different wavelengths of light) of light projected into a pregnant mammal's abdomen may be used to determine, for example, an impact of the pregnant mammal's abdominal tissue's interaction with light traveling through from her abdomen may lead to greater accuracy when calculating fetal hemoglobin oxygen saturation.

How much light reaches a fetus is often times not linearly related to how much light is projected into the pregnant mammal's abdomen. Each pregnant mammal and fetus combination is different in terms of the geometry of their respective tissue layers and/or intrinsic characteristics such as hemoglobin oxygen saturation and/or blood profusion through tissue, which makes approximations for how much light reaches a fetus or other one size fits all calculations or corrections for the calculation of fetal hemoglobin oxygen saturation often times inaccurate. Thus, calibrating calculations using physiological characteristics of a pregnant mammal and/or pregnant mammal/fetus combination may assist with more accurately calculating fetal hemoglobin oxygen saturation.

Transabdominal fetal oximetry and/or fetal pulse oximetry is often performed using near infrared (NIR) light. NIR light projected into a pregnant mammal's abdomen may be absorbed by, for example, the melanin in the pregnant mammal's skin, the pregnant mammal's myoglobin (muscle) tissue, and the hemoglobin in the pregnant mammal's blood, deoxygenated hemoglobin absorbs more light than oxygenated hemoglobin. Thus, knowing the how much melanin is in a pregnant mammal's skin, a concentration of her myoglobin layers, and/or her hemoglobin oxygen saturation can assist with predicting how much light, or photons, her hemoglobin is likely to absorb. Knowing this absorption characteristic (which may be expressed as an absorption coefficient (μₐ(λ)) in a mathematical equation—examples of which are provided herein) may make calculating the fetal hemoglobin oxygen saturation via, for example, one or more methods disclosed herein more accurate.

In addition, the intensity of light projected into the pregnant mammal's abdomen often decays exponentially with distance (in this case the distance between the maternal epidermis and the fetus' epidermis, or fetal depth) via, for example, the Inverse Square Law wherein the intensity of light incident on the fetus is proportional to the fetal depth.

NIR light projected into a pregnant mammal's abdomen may be scattered by, for example, adipose tissue present in the maternal abdomen and positioned between a fetal hemoglobin oxygen saturation probe and a fetus.

Thus, the factors of the pregnant mammal's melanin content, hemoglobin oxygen saturation, myoglobin concentration, and/or adipose tissue thickness may impact how much light is incident upon the fetus. It is important to understand one or more of these physiological characteristics of the pregnant mammal in order to understand how much light is incident on the fetus so that analysis of light reflected from the fetus and subsequent calculations of fetal hemoglobin oxygen saturation is accurate.

In some cases, calculations of hemoglobin oxygen saturation are performed using certain assumptions including, but not limited to, a pathlength for different wavelengths of light through tissue is the same (or so close as to have a negligible impact) and/or that light's scattering behavior as it passes through tissue is of negligible importance. While these assumptions may be appropriate for simplified applications (e.g., determining a user's hemoglobin oxygenation via projection of light through a finger or ear lobe), they may not always hold true (i.e., produce accurate results) when projecting light deeper into tissue as is the case when projecting light into a maternal abdomen in order to determine a hemoglobin oxygen saturation level for the pregnant mammal's fetus because, for example, a deeper probing geometry when probing a maternal abdomen may exaggerate the path-length difference for discordant wavelengths. Because these assumptions may not always hold true in this context, measurements or other calibration factors that factor in how layers of maternal tissue may impact light's behavior when passing through the tissue may improve the accuracy of determining hemoglobin oxygen saturation levels for a fetus. Examples of such measurements and/or calibration factors will be discussed below.

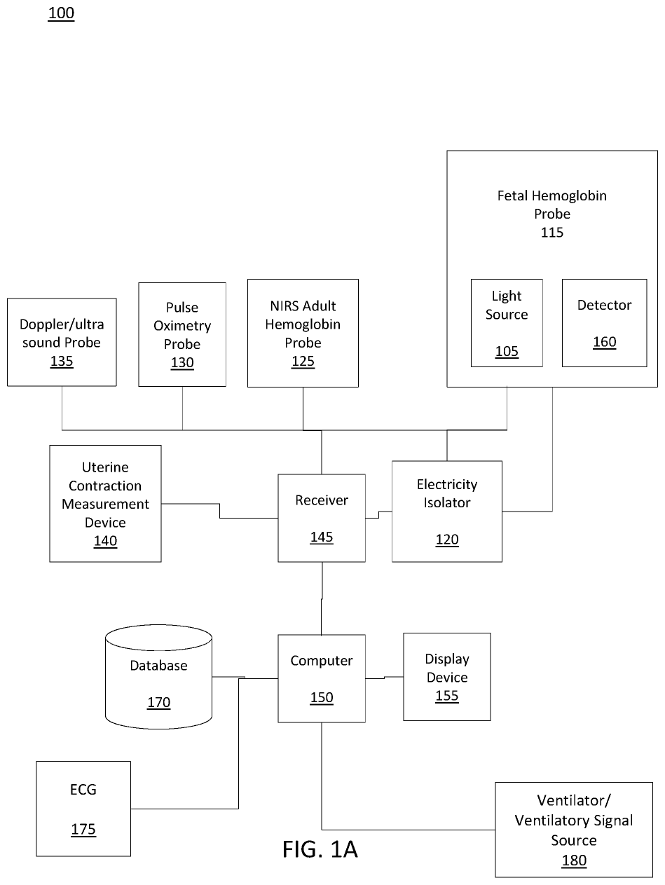

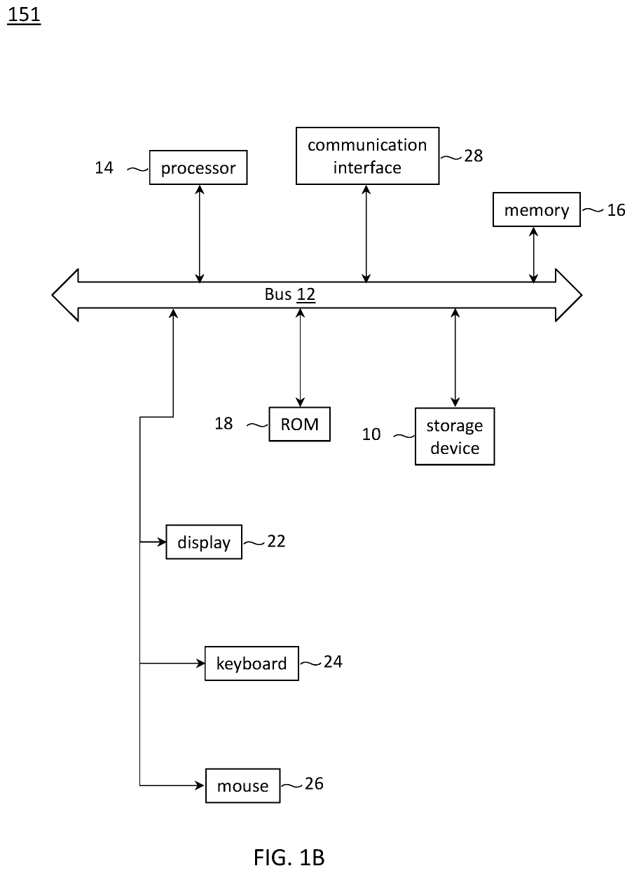

FIG. 1 provides an exemplary system 100 for detecting and/or determining fetal hemoglobin oxygen saturation levels. The components of system 100 may be coupled together via wired and/or wireless communication links. In some instances, wireless communication of one or more components of system 100 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

System 100 includes a light source 105 and a detector 160 that, at times, may be housed in a single housing, which may be referred to as fetal hemoglobin probe 115. Light source 105 may include a single, or multiple light sources and detector 160 may include a single, or multiple detectors.

Light sources 105 may transmit light at light of one or more wavelengths, including NIR, into the pregnant mammal's abdomen. Light sources 105 may be, for example, a LED, and/or a LASER, a tunable light bulb and/or a tunable LED that may be coupled to a fiber optic cable. On some occasions, the light sources may be one or more fiber optic cables optically coupled to a laser and arranged in an array. In some instances, the light sources 105 may be tunable or otherwise user configurable while, in other instances, one or more of the light sources may be configured to emit light within a pre-defined range of wavelengths. Additionally, or alternatively, one or more filters (not shown). These filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. In some examples, light source 105 may be configured so that it does not emit light that may, for example, irritate or burn the skin of the patient and/or harm the fetus. This may be achieved by, for example, configuring and/or instructing light source 105 to emit a high-intensity/high-power pulse of light for a short time duration. This high-intensity/high-power pulse of light may be used to, for example, improve a likelihood that detectors like detector 160 positioned relatively far away from the light source will receive sufficient light to detect following the light's transmission into the pregnant mammal's abdomen and emission therefrom in a manner that does not harm the pregnant mammal or her fetus. Additionally, or alternatively, one or more light source(s) 105 may be configured to emit light in a time division multiplexed manner so that, for example, signals received from each of a plurality of detectors, like detector 160, may be distinguished from one another. Light emitted in a time division multiplexed manner may be utilized for detectors that are relatively close to the light source(s) 105.

Detector 160 may be configured to detect a light signal emitted from the pregnant mammal and/or the fetus via, for example, transmission and/or back scattering. Detector 160 may convert this light signal into an electronic signal, which may be communicated to a computer or processor and/or an on-board transceiver that may be capable of communicating the signal to the computer/processor. This emitted light might then be processed in order to determine how much light, at various wavelengths, passes through the fetus and/or is reflected and/or absorbed by the fetal oxyhemoglobin and/or de-oxyhemoglobin so that a fetal hemoglobin oxygen saturation level may be determined. This processing will be discussed in greater detail below.

Exemplary detectors include, but are not limited to, cameras, traditional photomultiplier tubes (PMTs), silicon PMTs, avalanche photodiodes, and silicon photodiodes. In some embodiments, the detectors will have a relatively low cost (e.g., $50 or below), a low voltage requirement (e.g., less than 100 volts), and non-glass (e.g., plastic) form factor. In other embodiments, (e.g., contactless pulse oximetry) a sensitive camera may be deployed to receive light emitted by the pregnant mammal's abdomen. For example, detector 160 may be a sensitive camera adapted to capture small changes in fetal skin tone caused by changes in cardiovascular pressure associated with fetal myocardial contractions. In these embodiments, detector 160 and/or fetal hemoglobin probe 115 may be in contact with the pregnant mammal's abdomen, or not, as this embodiment may be used to perform so-called contactless pulse oximetry. In these embodiments, light sources 105 may be adapted to provide light (e.g., in the visible spectrum, near-infrared, etc.) directed toward the pregnant mammal's abdomen so that the detector 160 is able to receive/detect light emitted by the pregnant mammal's abdomen and fetus. The emitted light captured by detector 160 may be communicated to computer 150 for processing to convert the images to a measurement of fetal hemoglobin oxygen saturation according to, for example, one or more of the processes described herein.

A fetal hemoglobin probe 115, light source 105, and/or detector 160 may be of any appropriate size and, in some circumstances, may be sized so as to accommodate the size of the pregnant mammal using any appropriate sizing system (e.g., waist size and/or small, medium, large, etc.). Exemplary lengths for a fetal hemoglobin probe 115 include a length of 4 cm-40 cm and a width of 2 cm-10 cm. In some circumstances, the size and/or configuration of a fetal hemoglobin probe 115, or components thereof, may be responsive to skin pigmentation of the pregnant mammal and/or fetus. In some instances, the fetal hemoglobin probe 115 may be applied to the pregnant mammal's skin via tape or a strap that cooperates with a mechanism (e.g., snap, loop, etc.) (not shown). In some embodiments, fetal hemoglobin probe 115 may be configured as a multiparameter unit that may be configured to, for example, communicate both ways with, for example, computer 150 and/or a processor to, for example, integrate, share, and/or store data amongst the different components of system 100.

System 100 includes a number of optional independent sensors/probes designed to monitor various aspects of maternal and/or fetal health and may be in contact with a pregnant mammal. These probes/sensors are a NIRS adult hemoglobin probe 125, a pulse oximetry probe 130, a Doppler and/or ultrasound probe 135, and a uterine contraction measurement device 140. Not all embodiments of system 100 will include all of these components. In some embodiments, system 100 may also include an electrocardiography (ECG) machine (not shown) that may be used to determine the pregnant mammal's and/or fetus' heart rate and/or an intrauterine pulse oximetry probe (not shown) that may be used to determine the fetus' heart rate. The Doppler and/or ultrasound probe 135 may be configured to be placed on the abdomen of the pregnant mammal and may be of a size and shape that approximates a silver U.S. dollar coin and may provide information regarding fetal position, orientation, and/or heart rate. Pulse oximetry probe 130 may be a conventional pulse oximetry probe placed on pregnant mammal's hand and/or finger to measure the pregnant mammal's hemoglobin oxygen saturation. NIRS adult hemoglobin probe 125 may be placed on, for example, the pregnant mammal's 2nd finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (e.g., measured in e.g., mmHg) over time. In some instances, the uterine contraction measurement device 140 is and/or includes a zotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical impedance as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated to receiver 145 for communication to computer 150 and display on display device 155 and, in some instances, may be considered secondary signals. As will be discussed below, measurements provided by NIRS adult hemoglobin probe 125, pulse oximetry probe 130, a Doppler and/or ultrasound probe 135, uterine contraction measurement device 140 may be used in conjunction with fetal hemoglobin probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal hemoglobin probe 115, NIRS adult hemoglobin probe 125, pulse oximetry probe 130, Doppler and/or ultrasound probe 135, and/or uterine contraction measurement device 140. Communication of receiver 145 with other components of system may be made using wired or wireless communication.

In some instances, one or more of NIRS adult hemoglobin probe 125, pulse oximetry probe 130, a Doppler and/or ultrasound probe 135, uterine contraction measurement device 140 may include a dedicated display that provides the measurements to, for example, a user or medical treatment provider. It is important to note that not all of these probes may be used in every instance. For example, when the pregnant mammal is using fetal hemoglobin probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140) of system 100 may not be used.

In some instances, receiver 145 may be configured to process or pre-process received signals so as to, for example, make the signals compatible with computer 150 (e.g., convert an optical signal to an electrical signal), amplify a received signal, and/or improve signal to noise ratio (SNR) by, for example, performing Fast Fourier transforms (FFT), bandwidth narrowing, and/or phase correlation filtering. In some instances, receiver 145 may be resident within and/or a component of computer 150. In some embodiments, computer 150 may amplify or otherwise condition the received detected signal so as to, for example, improve the signal-to-noise ratio.

Receiver 145 may communicate received, pre-processed, and/or processed signals to computer 150. Computer 150 may act to process the received signals, as discussed in greater detail below, and facilitate provision of the results to a display device 155. Exemplary computers 150 include desktop and laptop computers, servers, tablet computers, personal electronic devices, mobile devices (e.g., smart phones), Internet of things (IoT) that may enable remote patient/pregnant mammal monitoring, and the like. Exemplary display devices 155 are computer monitors, tablet computer devices, and displays provided by one or more of the components of system 100. In some instances, display device 155 may be resident in receiver 145 and/or computer 150. Computer 150 may be communicatively coupled to database 170, which may be configured to store information regarding physiological characteristic and/or combinations of physiological characteristic of pregnant mammals and/or their fetuses, impacts of physiological characteristic on light behavior, information regarding the calculation of hemoglobin oxygen saturation levels, calibration factors, and so on. In some embodiments, database 170 may be local (e.g., coupled to computer 150) and/or remote (e.g., a cloud-computing database).

In some embodiments, a pregnant mammal may be electrically insulated from one or more components of system 100 by, for example, an electricity isolator 120. Exemplary electricity insulators 120 include circuit breakers, ground fault switches, and fuses.

System 100 may also include an electrocardiography (ECG) machine 175, and/or a ventilatory/respiratory signal source 180. ECG 175 may be used to determine the pregnant mammal's and/or fetus's heart rate. In some embodiments, ECG 175 may be a fetal ECG that is used internally via, for example, placement in the birth canal may be used to determine the fetus's heart rate.

In some embodiments, system 100 may include a ventilatory/respiratory signal source 180 that may be configured to monitor the pregnant mammal's respiratory rate and provide a respiratory signal indicating the pregnant mammal's respiratory rate to, for example, computer 150. Additionally, or alternatively, ventilatory/respiratory signal source 180 may be a source of a ventilatory signal obtained via, for example, cooperation with a ventilation machine. Exemplary ventilatory/respiratory signal sources180 include, but are not limited to, a carbon dioxide measurement device, a stethoscope and/or electronic acoustic stethoscope, a device that measures chest excursion for the pregnant mammal, and a pulse oximeter. A signal from a pulse oximeter may be analyzed to determine variations in the PPG signal that may correspond to respiration for the pregnant mammal. Additionally, or alternatively, ventilatory/respiratory signal source 180 may provide a respiratory signal that corresponds to a frequency with which gas (e.g., air, anesthetic, etc.) is provided to the pregnant mammal during, for example, a surgical procedure. This respiratory signal may be used to, for example, determine a frequency of respiration for the pregnant mammal.

In some embodiments, measurements provided by NIRS adult hemoglobin probe 125, pulse oximetry probe 130, a Doppler and/or ultrasound probe 135, uterine contraction measurement device 140, ECG 175, and/or ventilatory/respiratory signal source 180 may be used in conjunction with fetal probe 115 to isolate a fetal pulse signal and/or fetal heart rate from a maternal pulse signal and/or maternal heart rate.

FIG. 1B provides an example of a processor-based system 151 that may store and/or execute instructions for the processes described herein. Processor-based system 151 may be representative of, for example, computing device 150. Note, not all of the various processor-based systems which may be employed in accordance with embodiments of the present invention have all of the features of system 151. For example, certain processor-based systems may not include a display inasmuch as the display function may be provided by a client computer communicatively coupled to the processor-based system or a display function may be unnecessary. Such details are not critical to the present invention.

System 151 includes a bus 12 or other communication mechanism for communicating information, and a processor 14 coupled with the bus 12 for processing information. System 151 also includes a main memory 16, such as a random-access memory (RAM) or other dynamic storage device, coupled to the bus 12 for storing information and instructions to be executed by processor 14. Main memory 16 also may be used for storing temporary variables or other intermediate information during execution of instructions to be executed by processor 14. System 151 further includes a read only memory (ROM) 18 or other static storage device coupled to the bus 12 for storing static information and instructions for the processor 14. A storage device 10, which may be one or more of a hard disk, flash memory-based storage medium, a magnetic storage medium, an optical storage medium (e.g., a Blu-ray disk, a digital versatile disk (DVD)-ROM), or any other storage medium from which processor 14 can read, is provided and coupled to the bus 12 for storing information and instructions (e.g., operating systems, applications programs and the like).

System 151 may be coupled via the bus 12 to a display 22, such as a flat panel display, for displaying information to a user. An input device 24, such as a keyboard including alphanumeric and other keys, may be coupled to the bus 12 for communicating information and command selections to the processor 14. Another type of user input device is cursor control device 26, such as a mouse, a trackball, or cursor direction keys for communicating direction information and command selections to processor 14 and for controlling cursor movement on the display 22. Other user interface devices, such as microphones, speakers, etc. are not shown in detail but may be involved with the receipt of user input and/or presentation of output.

The processes referred to herein may be implemented by processor 14 executing appropriate sequences of processor-readable instructions stored in main memory 16. Such instructions may be read into main memory 16 from another processor-readable medium, such as storage device 10, and execution of the sequences of instructions contained in the main memory 16 causes the processor 14 to perform the associated actions. In alternative embodiments, hard-wired circuitry or firmware-controlled processing units (e.g., field programmable gate arrays) may be used in place of or in combination with processor 14 and its associated computer software instructions to implement the invention. The processor-readable instructions may be rendered in any computer language.

System 151 may also include a communication interface 28 coupled to the bus 12. Communication interface 28 may provide a two-way data communication channel with a computer network, which provides connectivity to the plasma processing systems discussed above. For example, communication interface 28 may be a local area network (LAN) card to provide a data communication connection to a compatible LAN, which itself is communicatively coupled to other computer systems. The precise details of such communication paths are not critical to the present invention. What is important is that system 151 can send and receive messages and data through the communication interface 28 and in that way communicate with other controllers, etc.

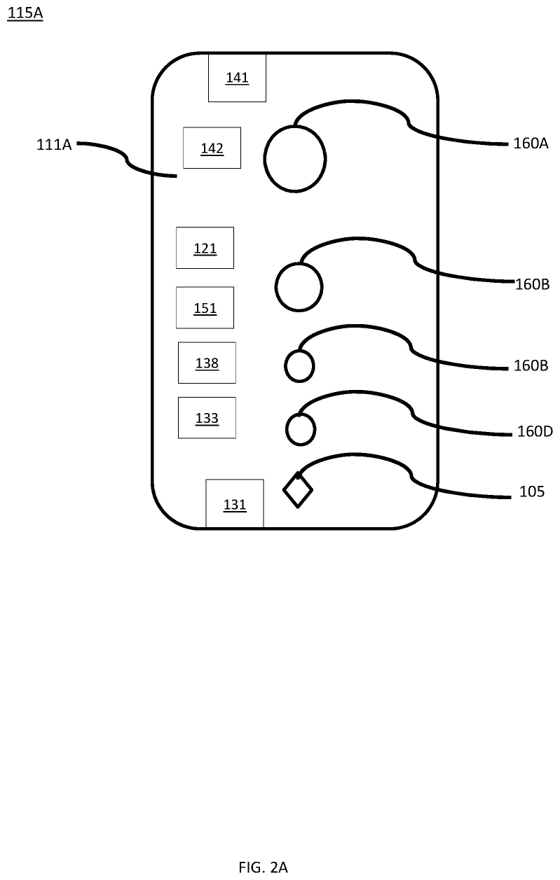

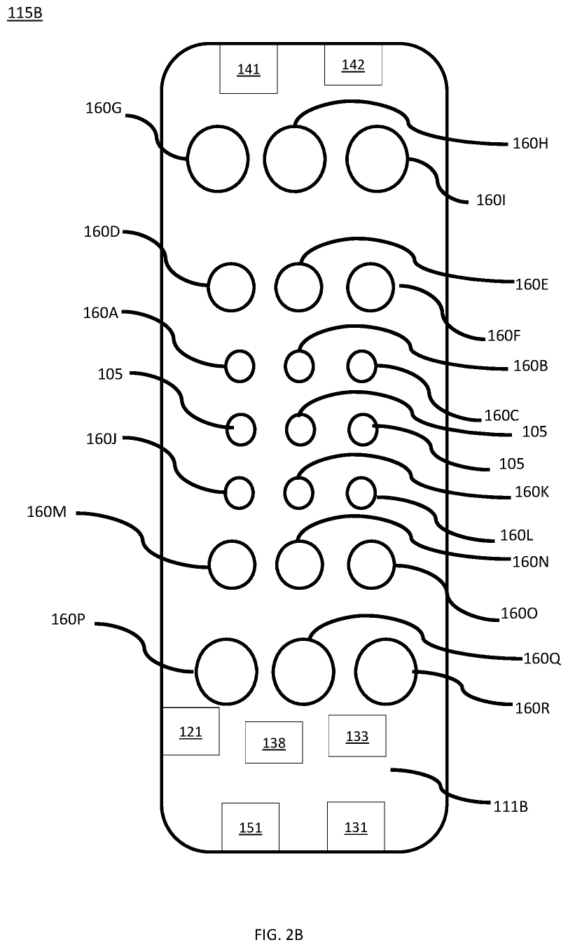

FIG. 2A is a block diagram illustrating an exemplary fetal probe 115A with housing 111A that houses a light source 105 and a plurality of detectors 160A-160D arranged in an exemplary array. Housing 111A may be any housing configured to house components of fetal probe 115A including light sources 105, the plurality of detectors 160A-160D, an optional power source 121 (e.g., a battery), a fetal depth probe 138, a maternal probe 133, a communication device (e.g., antenna or transceiver) 142, a processor 151, a power port 141, and/or a communication port 131. Exemplary fetal probe 115A includes a light source 105 substantially aligned with along the Y-axis with four detectors 160A-160D. In some embodiments, the gain, or sensitivity, of a detector 160A-160D may vary with its position relative to light source 105 so that, for example, detectors positioned further away from light source 105 (e.g., detectors 160A and 160B) have a greater gain/sensitivity than detectors positioned closer to light source 105 (e.g., detectors 160C and 160D).

In one example, fetal probe 115A may include a light source 105 configured to emit light of a plurality of wavelengths such as 735 nm, 760 nm, 810 nm, 808 nm, and/or 850 nm and each of detectors 160A-160D may be configured to detect light/photons of each of these wavelengths. An exemplary distance between light source 105 and detector 160A is 3 cm, an exemplary distance between light source 105 and detector 160B is 5 cm, an exemplary distance between light source 105 and detector 160C is 7 cm, and an exemplary distance between light source 105 and detector 160D is 10 cm.

FIG. 2B is a block diagram illustrating an exemplary fetal probe 115B with a plurality of light sources 105 and detectors 160A-160U arranged in an exemplary array within a housing 111B. Housing 111B may be any housing configured to house components of fetal probe 115B including the plurality of light sources 105, the plurality of detectors 160A-160U, optional power source 121 (e.g., a battery), fetal depth probe 138, maternal pulse oximetry probe 133, communication device (e.g., antenna or transceiver) 142, processor 151, power port 141, and/or communication port 131. Exemplary fetal probe 115A includes a light source 105 substantially aligned with along the Y-axis with four detectors 160A-160D.

Exemplary fetal probe 115B includes a row of three light sources 105 positioned in the approximate center, along the Y-axis, of housing 111B. The plurality of light sources 105 may be substantially aligned with one another along the X-axis. Housing may further include nine detectors 160A-1601 positioned above the light sources 105 in three rows with three columns and nine detectors 160K-160R positioned below the light sources 105 in three rows with three columns each. In some embodiments, the gain, or sensitivity, of a detector 160E-160R may vary with its position relative to a light source 105 so that, for example, detectors positioned further away from light source 105 have a greater gain/sensitivity as explained above with regard to fetal probe 115A.

The arrangement sources and detectors of FIGS. 2A and 2B are provided by way of example only and is not intended to limit an arrangement and/or number of light sources 105 and/or detectors 160 that may be used. Any arrangement thereof may be used to detect optical signals and convert them into the detected electronic signal(s) discussed herein.

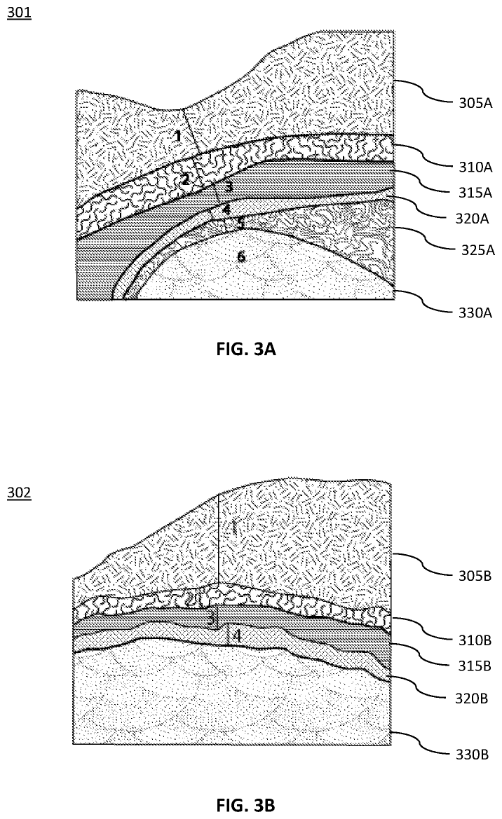

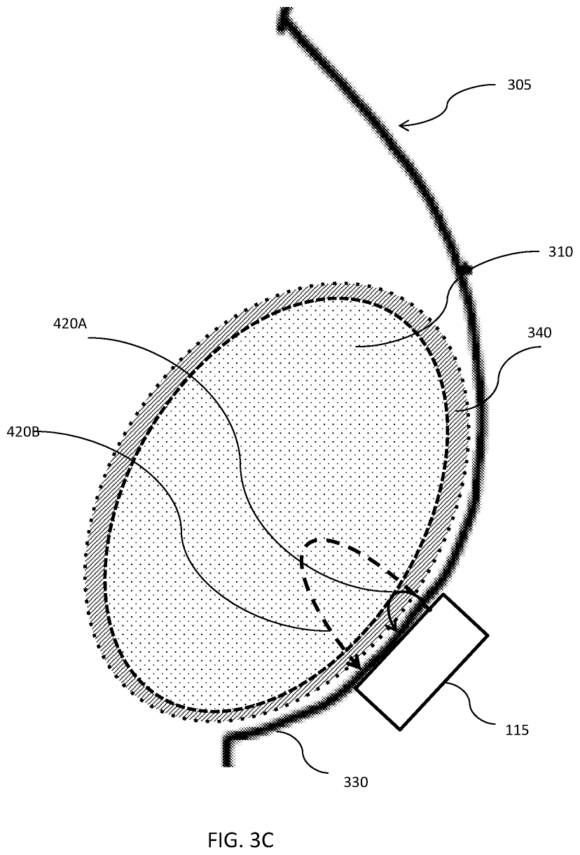

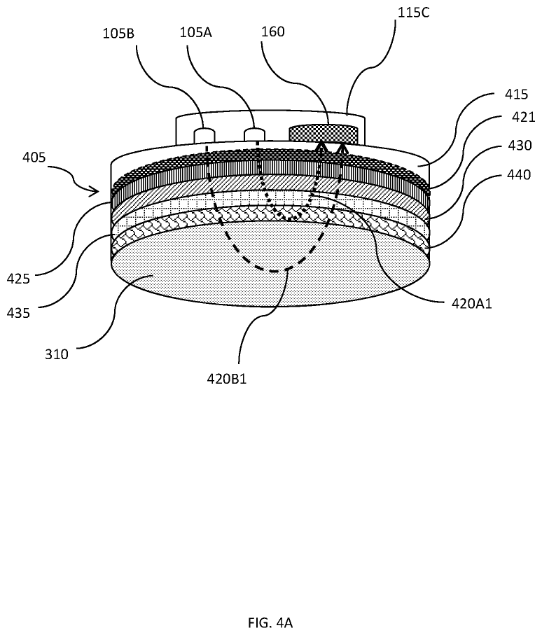

FIGS. 3A and 3B provide illustrations 301 and 302, respectively, of some layers of tissue present in two different maternal abdomens with their respective fetuses included in the illustration. Information used to generate illustrations 301 and 302 may be received from, for example, ultrasound imaging devices (e.g., Doppler/ultrasound probe 135) and/or MRI images.

Illustrations 301 and 302 provide exemplary dimensions for some layers of maternal tissue positioned proximate to a placement of a fetal hemoglobin probe 115 as well as the fetus including a depth of the fetus within the respective pregnant mammal's abdomen. A depth of a fetus may be understood as, for example, a distance between the epidermis of the pregnant mammal and the epidermis of the fetus and/or the aggregate width of the layers of maternal tissue and amniotic fluid. Illustration 301 shows maternal abdominal tissue for a fetus that has reached 29 weeks of gestation. The layers of tissue shown in illustration 301 include a subcutaneous fat layer 305A, an abdominal muscle (skeletal muscle) layer 310A, an intraperitoneal fat layer 315A, a uterine wall (smooth muscle) layer 320A, an amniotic fluid layer 325A, and a fetus 330A. Measurements for a width of each of these layers and are taken at a position proximate to (e.g., underneath) fetal hemoglobin probe 115. The approximate location for where width measurements are taken is represented by a line connecting a top and bottom of the layer of interest. For example, in FIG. 3A, a width of subcutaneous fat layer 305A is represented by line 1, a width of abdominal muscle layer 310 is represented by line 2, a width of intraperitoneal fat layer 315A is represented by line 3, a width of uterine wall layer 320A is represented by line 4, and a width of amniotic fluid layer 325A is represented by line 5. Approximate dimensions for these layers of maternal tissue that are positioned proximate to (e.g., underneath) fetal hemoglobin probe 115 are:

Subcutaneous fat layer 305A: 10.2 mm (represented by line 1);

Abdominal muscle layer 310A: 7.1 mm (represented by line 2);

Intraperitoneal fat layer 315A: 2.0 mm (represented by line 3);

Uterine wall layer 320A: 3.1 mm (represented by line 4);

Amniotic fluid layer 325A: 3.6 mm (represented by line 5); and

Fetus 330A.

A total distance from the maternal epidermis to the epidermis of fetus 330A (i.e., fetal depth) in this example is 28 mm.

The fetus shown in illustration 302 of FIG. 3B has reached 35 weeks of gestation. The layers of tissue shown in illustration 302 include a subcutaneous fat layer 305B, an abdominal muscle (skeletal muscle) layer 310B, an intraperitoneal fat layer 315B, a uterine wall (smooth muscle) layer 320B, and a fetus 330B. Measurements for a width of each of these layers and are taken at a position proximate to (e.g., underneath) fetal hemoglobin probe 115. The approximate location for where width measurements are taken is represented by a line connecting a top and bottom of the layer of interest. For example, in FIG. 3B, a width of subcutaneous fat layer 305B is represented by line 1, a width of abdominal muscle layer 310 is represented by line 2, a width of intraperitoneal fat layer 315B is represented by line 3, and a width of uterine wall layer 320B is represented by line 2.

Approximate dimensions for the layers of maternal tissue that are positioned proximate to (e.g., underneath) fetal hemoglobin probe 115 are:

Subcutaneous fat layer 305B: 11.3 mm (represented by line 1);

Abdominal muscle layer 310B: 3.1 mm (represented by line 2);

Intraperitoneal fat layer 315B: 3.1 mm (represented by line 3);

Uterine wall layer 320B: 2.3 mm (represented by line 4); and

Fetus 330B.

A total distance from maternal skin to fetus (i.e., fetal depth) in this example is 19.8 mm. Because the fetus is more developed and larger at 35 week's gestation, a width of the amniotic fluid is negligible and is not included in this example. In addition, for illustrations 301 and 302, a width of the skin of the pregnant mammal is also negligible at approximately 1-1.5 mm.

In some embodiments, the fetus 330A and/or fetal layer 330B may be divided into one or more additional layer(s) (not shown). These layers may pertain to, for example, one or more of vernix, hair, skin, bone, etc. In some embodiments, information regarding one or more of these layers (e.g., melanin content of fetal skin and/or hair color) may be deduced from, for example, parentage of the fetus, genetic testing of the fetus, and/or direct observation of the fetus via, for example, an optic scope and/or transvaginal examination.

FIG. 3C illustrates provides a midsagittal plane view of pregnant mammal's 305 abdomen with fetal hemoglobin probe 115 positioned thereon. As shown in FIG. 3, the pregnant mammal's abdomen 305 includes an approximation of a fetus 330, a uterus 340, and maternal tissue (e.g., skin, muscle, etc.) 330. Fetal hemoglobin probe 115 may be positioned anywhere on the pregnant mammal's abdomen and, in some instances, more than one fetal hemoglobin probe 115 may be placed on the pregnant mammal's abdomen. FIG. 3C also shows a first optical signal 420A being projected into the pregnant mammal's abdomen where the depth of penetration of first optical signal 420A is only to the edge of the uterine wall 340 and then is back scattered, or transmitted through, into a detector of fetal hemoglobin probe 115 like detector 160. FIG. 3C further shows a second optical signal 420B being projected into the pregnant mammal's abdomen and penetrates fetus 330 prior to being detected by detector 160. First optical signal 420A may include light of a single wavelength or a plurality of wavelengths that may be, for example, red or NIR. In some embodiments, first optical signal may include light of two distinct wavelengths or ranges of wavelengths, one red and one NIR. Second optical signal 420B may include light of a single wavelength or a plurality of wavelengths that may be, for example, red or NIR. The wavelength(s) of second optical signal 420B may be different from those of first optical signal 420A and/or projected into the pregnant mammal's abdomen at different times so that second optical signal 420B may be distinguished from first optical signal 420A during processing of detected portions of first and second optical signals 420A and 420B, respectively. In some embodiments, first and second optical signals 420A and 420B may include light of two distinct wavelengths or ranges of wavelengths, one red and one NIR that are slightly different from one another. For example, first optical signal 420A may be red and second optical signal 420B may be NIR, both first and second optical signals 420A and 420B may be red or NIR. In these examples, the wavelengths of first and second optical signals 420A and 420B may be selected so that any differences in their respective path lengths will be negligible. The two wavelengths may enable pulse oximetry calculations using, for example, differences in absorption, or (μₐ(λ)), and/or scattering (μₛ(λ)) of the optical signal using, for example, the Lambert-Beer or modified Lambert-Beer calculations as, for example, described herein.

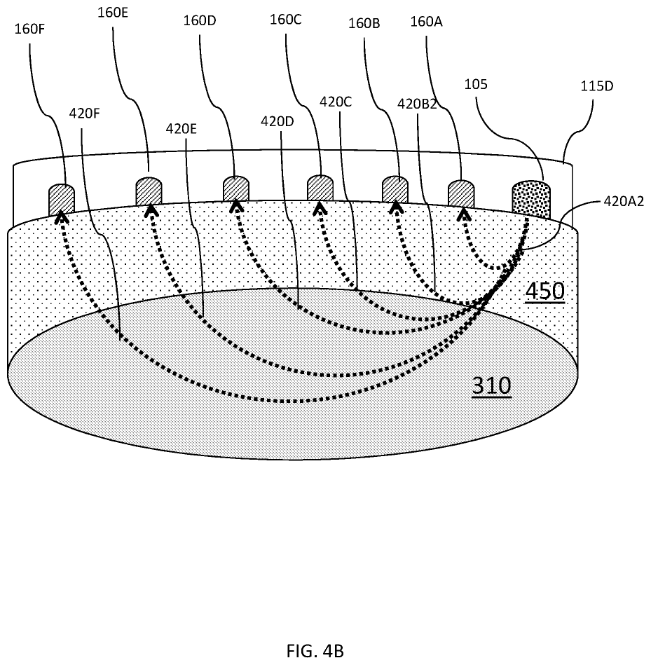

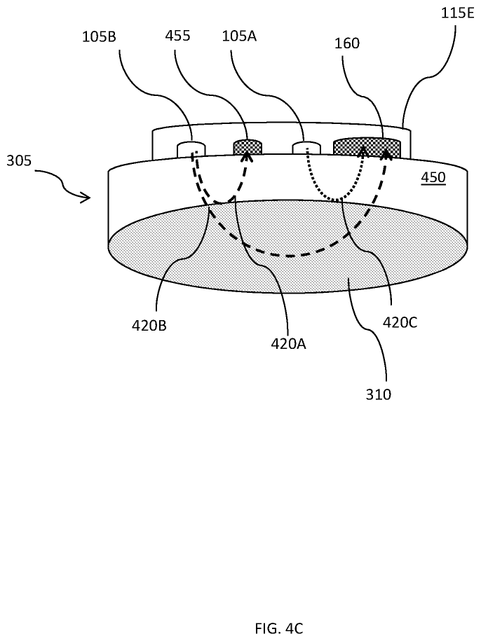

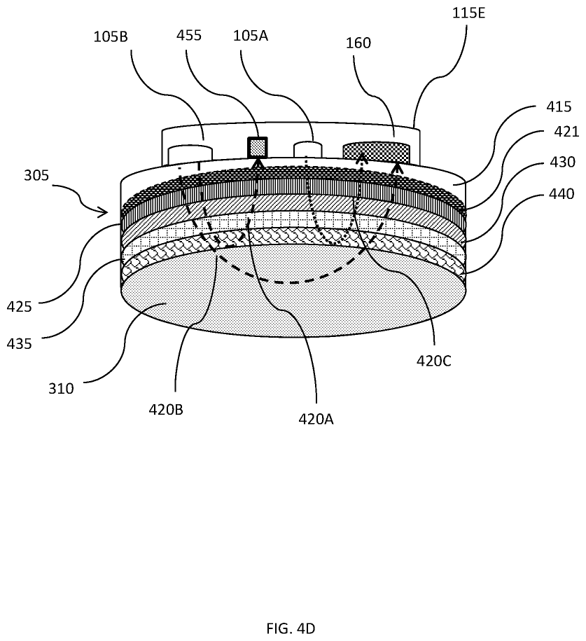

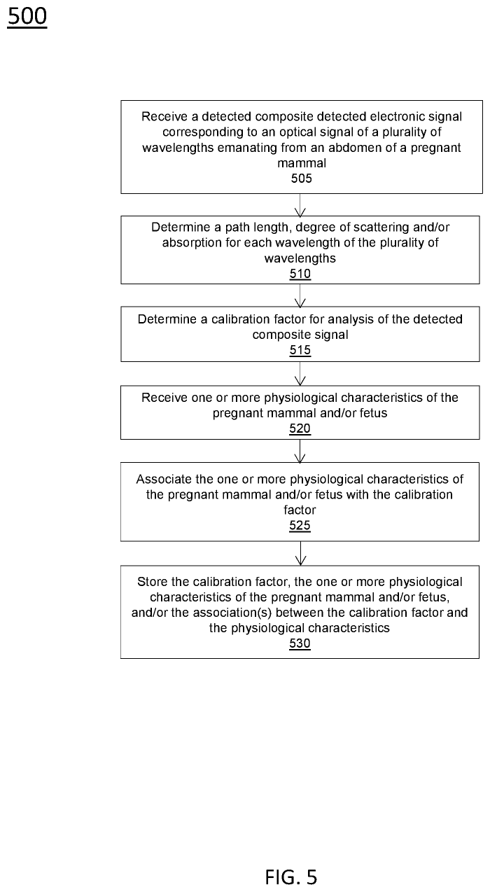

FIG. 4A illustrates an exemplary fetal hemoglobin probe 115C in contact with a pregnant mammal's abdomen in a manner similar to that shown in FIG. 3. FIG. 4A also shows a plurality of layers of tissue. More specifically, FIG. 4A shows a first layer that represents a maternal skin layer 415, a second layer that represents a maternal subcutaneous fat layer 421, a third layer that represents a maternal abdominal muscle (skeletal muscle) layer 425, a fourth layer that represents a maternal intraperitoneal fat layer 430, a fifth layer that represents a uterine wall (smooth muscle) layer 435, a sixth layer that represents an amniotic fluid layer 440, and a seventh layer that represents the fetus 330.

Fetal hemoglobin probe 115C includes a first light source 105A that emits first light beam 420A1, a second light source 105B that emits second light beam 420, and a detector 160. First and/or second light beams 420A1 and/or 420B1 may include light of a single, or multiple, wavelengths and may be within, for example, the red, NIR, or infra-red spectrum. In some circumstances, characteristics of light beam 420A1 may be different from the wavelength of light beam 420B1 and/or may be projected into the pregnant mammal's abdomen at a different time to enable distinguishing light projected from the two light sources when it is received by detector 160 and processed according to one or more of the processes described herein. In some embodiments, fetal hemoglobin probe 115C may include a filter (not shown) for detector 160 that may be attenuated to so that detector 160 detects and equal amount of light from first and second light sources 105A and 105B.

In many instances, a depth of light propagation through the pregnant mammal's abdomen is dependent on a distance between a light source and a detector. In some embodiments, the position of first light source 105A and/or second light source 105B may be adjusted (e.g., moved closer to, or further away from, detector 160) so as to, for example, adjust a depth of penetration for the light emitted therefrom. The adjustment may be facilitated by, for example, a track or other positioning device included in fetal hemoglobin probe 115C (not shown). In some instances, the positioning of first light source 105A and/or second light source 1056 may be adjusted responsively to a depth of fetus 330 within the pregnant mammal's abdomen (i.e., a measurement of the width of maternal tissue 405 positioned between the fetal hemoglobin probe 115C and the fetus 330). A measurement of a depth of fetus 330 within the pregnant mammal's abdomen may be provided by, for example, an ultrasound or Doppler probe like Doppler/ultrasound probe 135 and/or an MRI image, an illustration of a portion of which is shown in illustrations 301 and 302.

In some embodiments, first light source 105A may be positioned relative to detector 160 so that light emitted from first light source (i.e., light beam 420A1) only propagates through the maternal tissue 405 and does not reach fetus 330. Second light source 1056 may be positioned further away (relative to first light source 105A) from detector 160 so that light projected by second light source 105B (i.e., light beam 420B1) projects deeper into the pregnant mammal's abdomen than light beam 420A1 and back scattering therefrom and/or transmission therethrough are detected by detector 160. Stated differently, light source 105A may be positioned so light beam 420A1 only projects into maternal tissue 405 so that the portion of light beam 420A1 detected by detector 160 may only be back scattered from and/or transmitted through from maternal tissue 405 and not the fetus 330 while light source 105B may be positioned so light beam 420B1 projects into both maternal tissue 405 and fetus 330 so that the portion of light beam 420B1 detected by detector 160 may be back scattered from and/or transmitted through from maternal tissue 405 and the fetus 330. This positioning of first light source 105A may facilitate short separation (SS) measurements and the path of first light beam 420A1 and/or the detected amounts of first light beam 420A1 by detector 160 may be referred to herein as a SS channel. This positioning of second light source 105B may facilitate long separation (LS) measurements and the path of second light beam 420B1 and/or the detected amounts of second light beam 420B1 by detector 160 may be referred to herein as a LS channel.

FIG. 4B illustrates an exemplary fetal probe 115B positioned on a pregnant mammal's abdomen. The maternal tissue of the pregnant mammal's abdomen is represented as maternal tissue 405 and a fetus within the pregnant mammal's abdomen is represented as fetus 410.

Fetal probe 115D has one light source 105 and six detectors 160A, 160B, 160C, 160D, 160E, and 160F, each of which have a different position relative to source 105 with first detector 160 A being the closest to source 105 and sixth detector 160F being the furthest away from source 105. A position of a detector 160A-160F relative to source 105 may be referred to herein as a source/detector distance. In some examples, detectors 160A-160F may be arranged linearly and may be positioned 1 cm apart from one another so that first detector 160A is positioned 1 cm away from source 105, second detector 160B is positioned 1 cm away from first detector 160A, third detector 160C is positioned 1 cm away from second detector 160B, fourth detector 160D is positioned 1 cm away from third detector 160C, fifth detector 160E is positioned 1 cm away from fourth detector 160D, and sixth detector 160F is positioned 1 cm away from fifth detector 160E.

Source 105 may project an optical signal 420 into the pregnant mammal's abdomen 405 and a resultant optical signal may be detected by one or more of detector(s) 160A-160F. It is expected that the detectors positioned closer to source 105 will detect a portion of the optical signal that has been incident on the pregnant mammal's abdomen 405 but not fetus 330 and, in some embodiments, first detector 160A and/or second detector 160B may be positioned via, for example, setting of a source/detector distance, so that a majority, if not all, of an optical signal 420A2 and 420B1 detected by first and second detectors 160A and 160B, respectively, has only been incident of the pregnant mammal's abdomen 405 (i.e., is not incident on the fetus). Third-sixth detectors 160C-160F may detect portions of the optical signal 420C, 420D, 420E, and 420F that are incident on the pregnant mammal 405 and fetus 330 as shown in FIG. 4. In some cases, third detector 160C may be positioned 3-5 cm away from the light source and sixth detector 160F may be positioned 6-10 cm away from the light source. Additionally, or alternatively, third-sixth detectors 160C-160F may be positioned within 4-10 cm of the light source.

As the source/detector distance increases a proportion of the optical signal that corresponds to light that was incident on fetus 330 increases. Thus, optical signal 420F may include a higher proportion of light that was incident on the fetus than, for example, optical signal 420E or 420D.

FIGS. 4C and 4D illustrate an exemplary fetal probe 115E in contact with a pregnant mammal's abdomen in a manner similar to that shown in FIGS. 4A and 4B with layers of maternal tissue similar to those shown in FIG. 4A. The embodiment shown in FIG. 4C utilizes the simplified layer of maternal tissue 450 and the embodiment shown in FIG. 4D shows many layers of maternal tissue with layers of maternal tissue similar to those shown in FIG. 4A and fetal probe 115E is configured to enable double short separation (SS) analysis of light back scattered from and/or transmitted through the pregnant mammal's abdomen and the fetus contained therein.

Fetal probe 115E includes a first light source 105A that emits first optical signal 420C, a small detector 455, a second light source 105B that emits second optical signal 420, and detector 160. A first portion of second optical signal 420A3 may be detected by small detector 455 and a second portion of second optical signal 420B may be detected by detector 160. First and/or second light beams 420C and/or 420 may include light of a single, or multiple, wavelengths and may be within, for example, the red, near infra-red, and/or broadband spectrum. In some circumstances, the wavelength for optical signal 420C may be different from the wavelength of optical signal 420 and/or may be projected at different times to enable differentiation between light projected from the two light sources when it is received by detector 160 and processed according to one or more of the processes described herein. Small detector 455 may be similar to detector 160 but may have, for example, a smaller size and/or decreased sensitivity. In some instances, small detector 455 may be a small fiber detector. In some embodiments, fetal probe 115E may include a filter (not shown) for detector 160 that may be attenuated to so that detector 160 detects and equal amount of light from first and second light sources 105A and 105B.

In some embodiments, the position of first light source 105A and/or second light source 105B may be adjusted (e.g., moved closer to, or further away from, detector 160) so as to, for example, adjust a depth of penetration for the light emitted therefrom that is detected by detector 160. The adjustment may be facilitated by, for example, manual manipulation and/or placement of a detector and/or moving a detector along a track or other positioning device included in and/or associated with fetal probe 115E (not shown). In some instances, the positioning of first light source 105A and/or second light source 105B may be adjusted responsively to a depth of fetus 330 within the pregnant mammal's abdomen (i.e., a measurement of the width of maternal tissue 450 positioned between the fetal probe 115E and the fetus 330). A measurement of a depth of fetus 330 within the pregnant mammal's abdomen may be provided by, for example, an ultrasound or Doppler probe like Doper/ultrasound probe 135 and/or an image of the pregnant mammal's abdomen, illustrations of which are shown in FIGS. 3A and 3B.

In some embodiments, first light source 105A may be positioned relative to detector 160 so that light emitted from first light source (i.e., optical signal 420C) only propagates through the maternal tissue 305 and does not reach fetus 330. Second light source 105B may be positioned further away (relative to first light source 105A) from detector 160 so that light projected by second light source 105B (i.e., optical signal 420) projects deeper into the pregnant mammal's abdomen than optical signal 420C so that it reaches fetus 330 so that light back scattered from and/or transmitted through the fetus may be detected by detector 160. Small detector 455 may be positioned between first and second light sources 105A and 105B so that light (i.e., optical signal 420) only propagates through the maternal tissue 450 prior to detection by small detector 455 and does not reach fetus 330. This positioning of first light source 105A may facilitate collection of a first set of short separation (SS) measurements and the path of first optical signal 420C and/or the detected amounts of first optical signal 420C by detector 160 may be referred to herein as a first SS channel. This positioning of second light source 105B may facilitate long separation (LS) measurements and the path of second optical signal 420 and/or the detected amounts of second optical signal 420 by detector 160 may be referred to herein as a LS channel. This positioning of small detector 455 may facilitate a second set of short separation (SS) measurements and the path of first optical signal 420C and/or the detected amounts of first optical signal 420C by detector 160 may be referred to herein as a second SS channel. Thus, fetal probe 115E provides for SS measurements of both the first and second light sources 105A and 105B.

FIG. 5 provides a flowchart illustrating a process 500 for determining a fetal hemoglobin oxygen saturation level. Process 500 may be executed by, for example, any of the system or system components described herein.

Initially, a detected composite electronic signal may be received from a photo-detector (e.g., detector 160) by a processor and/or computer like computer 150 (step 505). The detected composite electronic signal may be received from, for example, a photo-detector, a transceiver coupled to the photo-detector, and/or a fetal hemoglobin probe such as fetal hemoglobin probe 115. The

The detected composite electronic signal may correspond to an optical signal of a plurality of wavelengths emanating (via, for example, transmission, back scattering, and/or reflection) from the abdomen of a pregnant mammal and/or her fetus. Light incident upon, and exiting from, the pregnant mammal's abdomen may be generated by one or more light sources, like light source 105 and may be of any acceptable frequency or wavelength (e.g., near infra-red (NIR)) and/or combination of frequencies and/or wavelengths. In some embodiments (e.g., when multiple detectors are used), the received detected composite electronic signals may include and/or be associated with a detector identifier (e.g., code) so that a position of a particular detected composite electronic signal may be known. This location may then be used to analyze the received detected composite electronic signals to determine various factors of the detected light and/or imaged tissue.

In step 510, a pathlength and/or a degree of scattering and/or absorption of each wavelength of light emanating from the pregnant mammal's abdomen may be measured and/or determined via, for example, analysis of the detected first optical signal. In some embodiments, execution of step 510 may include using the Modified Beer Lambert Law, which is reproduced below as Equation 1. Equation 1 may be used to, for example, determine an absorption coefficient for a particular wavelength (μₐ(λ)), a change in absorption coefficient, and/or an effective mean path length factor (DPF) for a particular wavelength (λ).

$$Δ {μ}_{a} ( λ ) = - \frac{1}{r * D P F ( λ )} \frac{Δ I ( λ )}{{I}_{0}} \qquad Equation 1$$

### Where:

Δμₐ(λ)=the change in an absorption coefficient for a given wavelength λ;

r=a distance between a light source and detector;

DPF=differential pathlength factor for the given wavelength λ;

ΔI=actual intensity of the light of the given wavelength λ as it changes over

the course of each heart beat pulse; and

Iₒ=incident intensity of the light of the given wavelength λ.

At times, Iₒ may be an average intensity measured over the time of trace (i.e., the time within which the measurement is taken and/or a composite electronic signal is detected). In some instances, values for Δμₐ(λ) and/or DPF may be a calibration factor (step 515) for other calculations described herein (e.g., equations 5, 6, 7a and/or 7b). At times, the DPF is estimated based on, for example, characteristics of light at wavelength λ. Additionally, or alternatively, DPF may be deduced from a spectral fitting of experimentally determined values for

$$\frac{Δ I ( λ )}{{I}_{0}} .$$

Additionally, or alternatively, DPF may be determined via measuring and/or calculating the amount scattering of light for a given wavelength. In some circumstances when, for example, directing light into a portion of the body that has an inhomogeneous geometry (such as the abdomen of a pregnant mammal), the DFP may be dependent on, for example, physical characteristics and/or intrinsic properties of the pregnant mammal and/or her fetus including, but not limited to, fetal depth, lipid concentration in tissue, width of layers of tissue, a location on the fetus being exposed to the light, a melanin content of the pregnant mammal and/or fetus, and so on. Additionally, or alternatively, the DFP may be dependent on a property of the detector such as a color of the sensor surface of the detector and/or a sensitivity of the detector.

In some embodiments, an absorption coefficient may be determined for different layers of maternal tissue (also referred to herein as individual absorption coefficients) using, for example, optical properties of the type of tissue and/or maternal geometry (e.g., width of a tissue layer) and/or tissue density using Equation 1. For example, a series of absorption coefficients for different types and/or features of maternal tissue may be experimentally-determined and/or modeled based on characteristics of the layers of maternal tissue. Exemplary characteristics include, but are not limited to, tissue type, tissue density, tissue layer thickness, position of the tissue layer (e.g., DFP and/or distance between source and detector (represented as r with regard to Equation 1)), and/or optical properties (e.g., scattering and/or absorption coefficients for known widths of tissue that may be modified, or otherwise adjusted, based on, for example, geometry of a particular pregnant mammal and/or fetus). In some embodiments, these individual absorption coefficients may then be aggregated together to generate a total absorption coefficient that more closely approximates the absorption of light for a particular situation (e.g., particular pregnant mammals, particular locations on a pregnant mammal's abdomen, etc.).

In some embodiments, steps 505-515 may be performed on an individual, per-pregnant mammal basis in order to, for example, tailor, or calibrate, instruments and/or calculations to account for, among other things, individual physiology, instrument (e.g. light source and/or detector) placement, noise, etc. Additionally, or alternatively, in other embodiments, steps 505-515 may be performed a plurality (e.g., hundreds, thousands, etc.) of times so that a plurality of calibration factors may be determined. In some cases, this plurality of determined calibration factors may be used to, for example, determine average calibration factors for pregnant mammals that may, or may not be associated with one or more of the pregnant mammal's physiological characteristics. For example, in one embodiment, calibration factors for 10,000 pregnant women may be determined. These calibration factors then may be used to determine, for example, a universal calibration factor for all pregnant women (e.g., average calibration factor for the 10,000 pregnant women) and/or may be grouped according to one or more physiological factors including, but not limited to, gestational age of the fetus, maternal weight, maternal height, maternal melanin content, etc.

Optionally, in step 520, one or more physiological characteristics, or parameters, of the pregnant mammal and/or fetus may be received and/or determined (via, for example, analysis of ultrasound information or an image of the pregnant mammal, illustrations of which are provided by FIGS. 3A and 3B). Exemplary physiological characteristics include, but are not limited to, melanin content and/or degree and/or type of skin pigmentation of woman's and/or fetus' skin, fetal depth, fetal gestational age, and/or a width of one or more layers of maternal or fetal tissue, abdominal wall thickness for the pregnant mammal, a percentage of hemoglobin blood concentration, a degree of blood perfusion in tissue which may be taken via, for example, a DC tissue measurement. In some cases, the physiological characteristics may be grouped as intrinsic or geometrical characteristics, wherein exemplary intrinsic characteristics are blood or tissue oxygenation and/or hemoglobin concentration levels and body mass index (BMI) abdominal wall thickness and/or a thickness of a layer of the abdominal wall may be geometrical characteristics.

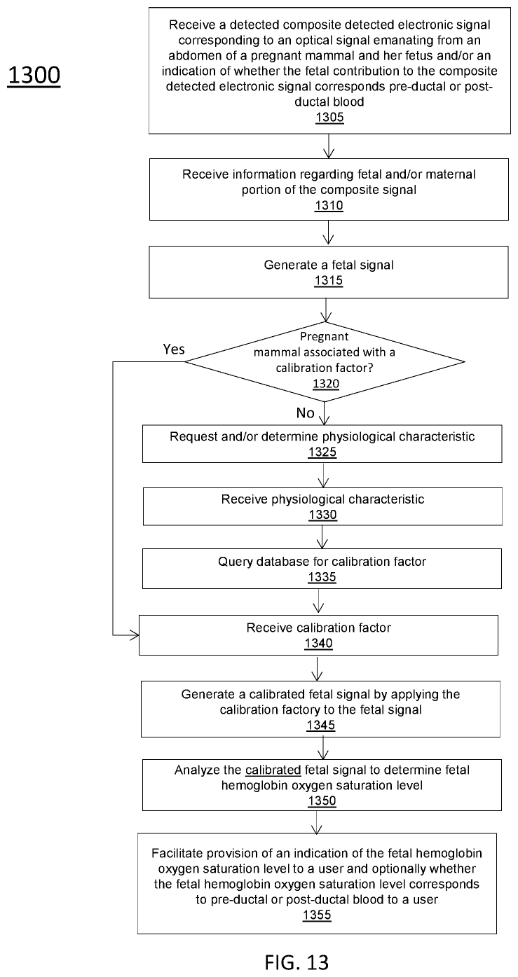

The one or more physiological characteristics of the pregnant mammal and/or fetus may be associated (e.g., indexed in a database table) with the calibration factor (step 525) in, for example, an index or look-up table. In some embodiments, these associations may be used to select a calibration factor that is appropriate for a particular pregnant mammal and/or fetus as will be discussed in greater detail below with regard to FIG. 13. The calibration factor, the one or more physiological characteristics of the pregnant mammal and/or fetus, and/or the association(s) therebetween may then be stored in, for example, a database like database 170 and/or a memory resident within a computer such as computer 150 (step 530). At times, calibration factors may be aggregated together and correlated to physiological properties; so that assumptions/calibration factors may be determined and/or applied without the need to individually calculate calibration factor for each pregnant mammal.

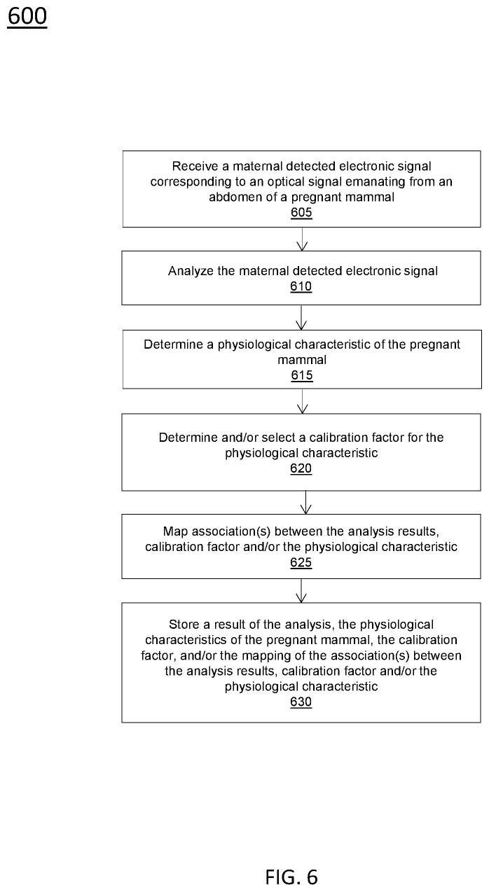

FIG. 6 provides a flowchart illustrating a process 600 for determining a fetal hemoglobin oxygenation saturation level using physiological characteristics of the pregnant mammal determined using a maternal detected electronic signal.

Process 600 may be performed by, for example, system 100 and/or components thereof. Process 600 may be executed in-situ during, for example, a labor and delivery of the fetus and/or a wellness checkup for the pregnant mammal. In some cases, process 600 may be executed on a continuous, periodic, and/or as-needed basis over a period of time such as the labor and delivery of the fetus so that, for example, the fetal hemoglobin oxygenation saturation level may be calibrated and recalibrated over time as needed and/or when conditions for the fetus and/or pregnant mammal change as may be the case when, for example, the fetus travels through the birth canal.

Initially, one or more maternal detected electronic signal(s) corresponding to an optical signal emanating from the abdomen of a pregnant mammal may be received (step 605). The optical signal may be generated by a light source like light source 105, incident on the pregnant mammal's abdomen, pass through a portion of the maternal abdominal tissue, and be reflected, or backscattered, through the maternal tissue where it is detected by a detector like detector 160. Exemplary optical signals like the optical signal that may be detected by a detector and received in step 605 are shown in FIG. 3C as first and second optical signals 325A and 420B.

In step 610, the maternal detected electronic signal may be analyzed to determine one or more physiological characteristics of the pregnant mammal (step 615). The analysis may be, for example, a frequency domain and/or time of flight analysis performed by a fetal hemoglobin probe configured to acquire time of flight measurements for photons projected into the maternal abdomen. In some embodiments, the determination of a physical characteristics for the pregnant mammal may be performed by determining how the pregnant mammal's tissue responds to incident light via measurement of, for example, absorption and/or scattering of the light in step 610. In some embodiments, the absorption and/or scattering of light may be expressed as an absorption coefficient or scattering coefficient, respectively, and may be used in one or more equations described herein. Then, in step 620, a calibration factor for the physiological characteristic may be determined (step 620). In some cases, steps 610 and 615 may be performed to determine one or more intrinsic physiological characteristics of the pregnant mammal that may be uniform across a portion of that pregnant mammal's abdomen. Such intrinsic physiological characteristics include, but are not limited to, melanin content and/or degree and/or type of skin pigmentation of woman's skin, fetal depth, a width of one or more layers of maternal or fetal tissue, abdominal wall thickness for the pregnant mammal, hemoglobin oxygen saturation, a degree of blood perfusion in abdominal tissue, whether the pregnant mammal is anemic, tissue oxygen saturation, and hemoglobin concentration levels.

A determination (i.e., calculation) and/or selection of a calibration factor in step 620 may be made by, for example, assessing how much light from the incident optical signal is absorbed and/or scattered by the pregnant mammal's abdominal tissue and/or a time of flight for photons of the optical signal to travel through the pregnant mammal's abdominal tissue and be detected by the detector. In some embodiments, the determination/selection of a calibration factor in step 620 may be performed by querying a database like database 170 and/or a memory resident within a computer such as computer 150 for calibration factors that correspond to the physiological characteristic determined in step 615. The database may be populated with correlated physiological characteristics and calibration factors via process 500. In some embodiments, associations between the results of executing steps 605, 610, 615, and/or 620 may be mapped to one another (step 625) and the results of executing steps 605, 610, 615, and/or 620 may be stored in, for example, a database like database 170 and/or a memory resident within a computer such as computer 150.

In one example, the physiological characteristic determined in step 615 may be a pregnant mammal's a skin color, pigmentation and/or melanin content, which may be determined by, for example determining how much of light incident on the pregnant mammal's abdomen is absorbed by the pregnant mammal's skin. The skin color of the pregnant mammal's skin may influence how much of the incident light is absorbed, which may impact how much of the light incident on the pregnant mammal's abdomen travels through the maternal tissue and is incident on the fetus may be associated with a known, or calculated, factor, which is determined and/or selected in step 620. For example, execution of 620 may include querying a database for calibration factors associated with how much light the pregnant mammal's skin absorbed. In this example, the calibration factor may be an absorption coefficient that may be associated with the pregnant mammal's absorption rate and/or skin color.

In another example, the maternal detected electronic signal received in step 605 may be analyzed in step 610 to determine the physiological characteristic a concentration or thickness of the myoglobin, or muscle, layers of the pregnant mammal's abdomen. This analysis may be performed using, for example, a fetal hemoglobin probe configured as a frequency domain NIRS system and/or a fetal hemoglobin probe configured as to obtain a time of flight for photons projected into the maternal abdomen that penetrate the maternal myoglobin layers and are reflected back to a detector. The myoglobin tissue of the pregnant mammal may absorb light projected into the maternal abdomen and a measurement and/or calculation of how much light that is absorbed (e.g., not detected) by the pregnant mammal's abdominal tissue may be used to determine the physiological characteristic of how thick and/or concentrated the maternal myoglobin layer(s) are. In step 620, this physiological characteristic may be used to, for example calculate a calibration factor and/or query a database of calibration factors to find a calibration factor associated with the physiological characteristic of the myoglobin thickness and/or concentration determined for the pregnant mammal.

In another example, the maternal detected electronic signal received in step 605 may be analyzed in step 610 to determine the physiological characteristic a total thickness of pregnant mammal's abdomen (which may also be referred to herein as fetal depth) which may vary over the course of a pregnancy due to the increasing size of the fetus. Additionally, or alternatively, the thickness of the pregnant mammal's abdominal tissue may vary of the course of the pregnancy and/or during labor and delivery of the fetus because of pre-eclampsia or eclampsia, which can cause edema that causes the pregnant mammal's abdominal thickness to change. The analysis of step 610 for this example may be performed using, for example, a fetal hemoglobin probe configured as a frequency domain NIRS system and/or a fetal hemoglobin probe configured as to obtain a time of flight for photons projected into the maternal abdomen that penetrate the maternal abdominal layers and are reflected back to a detector. The abdominal tissue of the pregnant mammal may absorb light projected into the maternal abdomen and a measurement and/or calculation of how much light that is absorbed (e.g., not detected) by the pregnant mammal's abdominal tissue may be used to determine the physiological characteristic of how thick the pregnant mammal's abdominal tissue is. In step 620, this physiological characteristic may be used to, for example calculate a calibration factor and/or query a database of calibration factors to find a calibration factor associated with the physiological characteristic of the myoglobin concentration and/or thickness determined for the pregnant mammal.

In yet another example, the maternal detected electronic signal received in step 605 may be analyzed in step 610 to determine the physiological characteristic a thickness of the adipose, or fat, layers of the pregnant mammal's abdomen. This analysis may be performed using, for example, a fetal hemoglobin probe configured as a frequency domain NIRS system and/or a fetal hemoglobin probe configured as to obtain a time of flight for photons projected into the maternal abdomen that penetrate the maternal adipose layers and are reflected back to a detector. The adipose tissue of the pregnant mammal may scatter light projected into the maternal abdomen and a measurement and/or calculation of how much light is scattered (e.g., not detected) by the pregnant mammal's adipose tissue may be used to determine the physiological characteristic of how thick the maternal adipose layer(s) are. In step 620, this physiological characteristic may be used to, for example calculate a calibration factor and/or query a database of calibration factors to find a calibration factor associated with the physiological characteristic of the adipose thickness determined for the pregnant mammal.

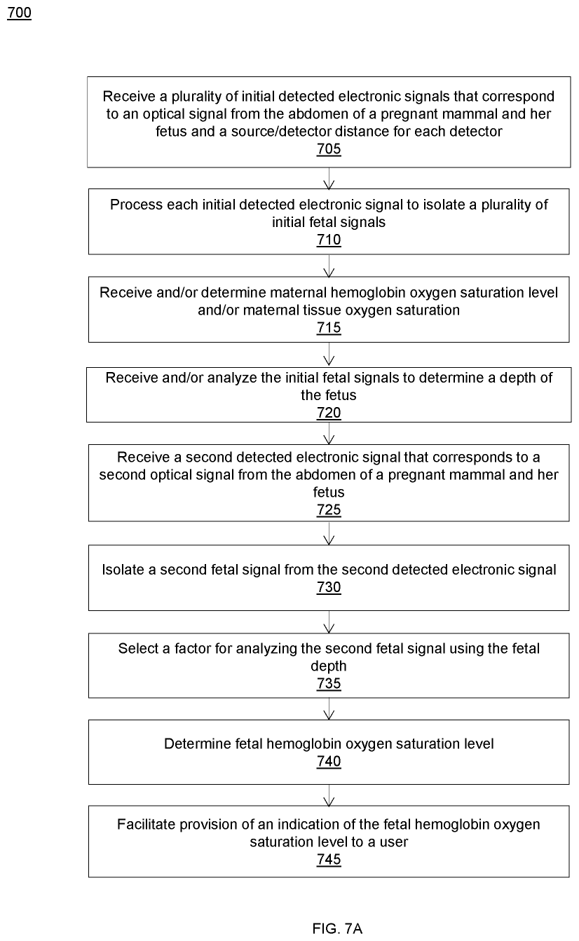

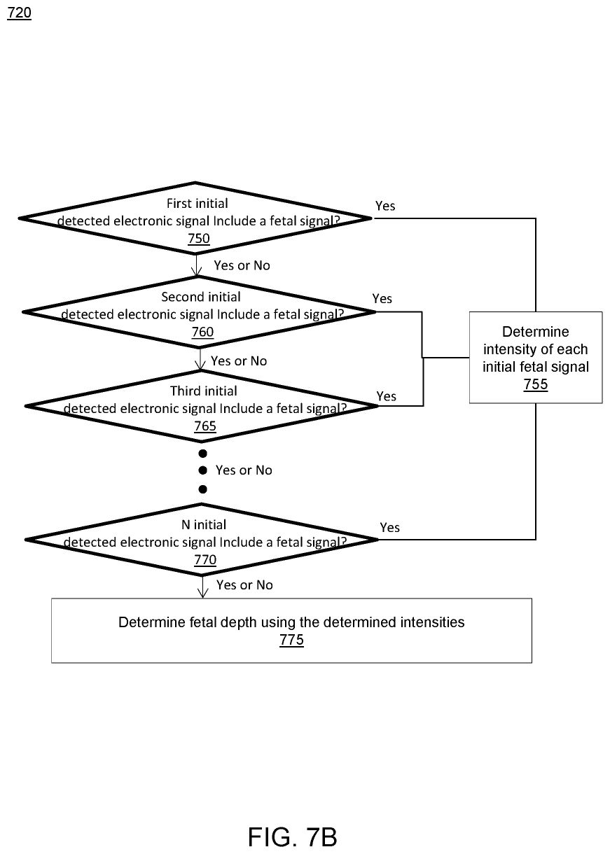

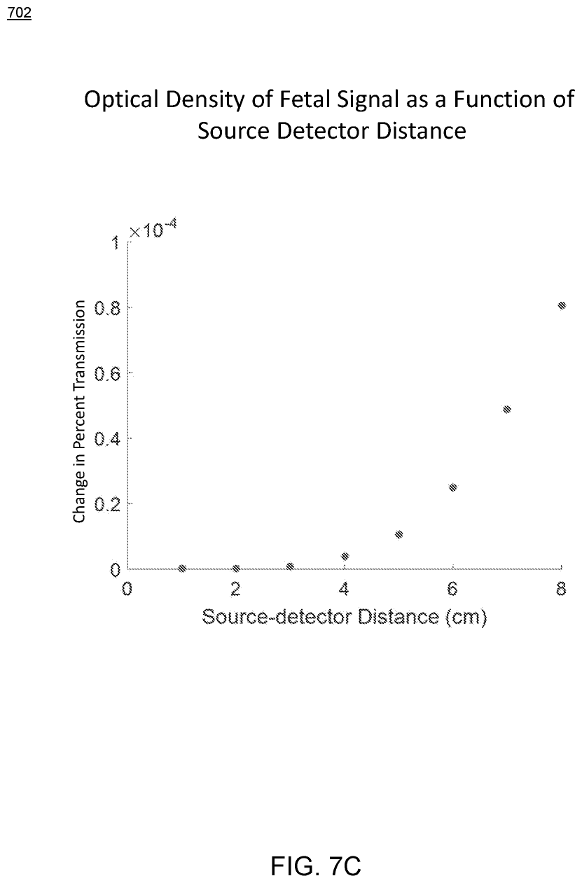

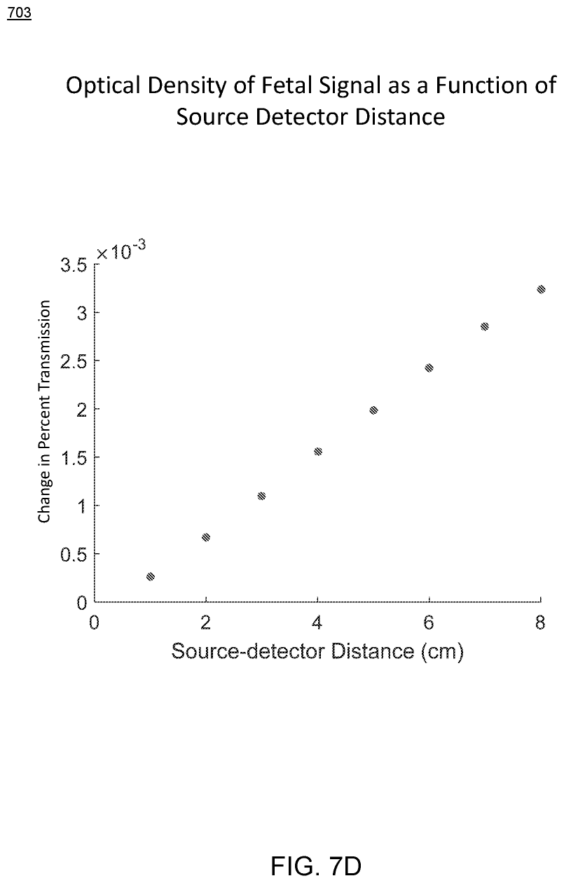

FIG. 7A is a flowchart illustrating a process 700 for determining a fetal depth and/or a level of oxygen saturation for fetal hemoglobin. Process 700 may be performed by, for example, system 100 and/or components thereof.

Optionally, in step 705, a plurality of first detected electronic signals, each of which correspond to an optical signal of one or more wavelengths projected into the pregnant mammal's abdomen by, for example, one or more light sources like light source 105 and exiting therefrom via, for example, reflection, back scattering, and/or transmission (i.e., passing through the maternal abdomen) may be received by, for example, a computer or processor such as computer 150. Each of the first detected signals of the plurality of first detected signals may be received from a different detector like detectors 160A-160V as shown and discussed above with regard to FIGS. 3A, 3B, and 3. The received first detected electronic signals may be associated with a detector identifier. Each detector may have a different source/detector distance. For example, a probe like fetal probe 115A, 115B, 115D, and/or 115E may have a source and a plurality of detectors with each detector having a different source/detector distance. In some embodiments, an optical signal detected by the detectors arranged further away from the source may have a higher proportion of light that was incident on the fetus than detectors arranged relatively close to the source.

An exemplary range of wavelengths for the optical signals that correspond to the first detected electronic signals is between 600 and 1000 nm and may be similar to one or more of optical signals 420. In some embodiments, the optical signal may be a broadband optical signal (e.g., white light and/or a range of, for example, 10, 15, or 20 wavelengths) and the received first detected signal may correspond to an optical signal of a plurality of wavelengths. In some embodiments, the optical signal, or a portion thereof, may be of a set, or known, wavelength that may be at an isosbestic point for light directed into human tissue to determine a ratio of oxygenated and de-oxygenated hemoglobin for the human's blood such as 808 nm. Light at this wavelength is reflected from oxygenated and de-oxygenated hemoglobin in the same way.

When step 705 is executed, each of the first detected electronic signals received in step 705 may be processed to isolate a portion thereof that corresponds to light that has been incident upon the fetus (step 710). This isolated portion of each of the first detected electronic signal may be referred to herein as a first fetal signal. Step 710 may be executed using any appropriate method of isolating a fetal signal from a corresponding first detected electronic signal including the methods disclosed herein. Appropriate methods include, but are not limited to, reducing noise in the signal via, for example, application of filtering or amplification techniques, determining a portion of the first detected electronic signal that is contributed by the pregnant mammal and then subtracting, or otherwise removing, that portion of the first detected electronic signal from the received first detected electronic signals and/or receiving information regarding fetal a heart rate and using that information to lock in (via, for example, a lock-in amplifier) on a portion of the received first detected electronic signals generated by the fetus.

Optionally, execution of step 710 may include pre-processing one or more of the first detected electronic signals in order to, for example, remove noise from the signal and/or confounding effects of the pregnant mammal's anatomy or physiological signals (e.g., a respiratory signal) from the first detected electronic signals. Execution of the pre-processing may include, but is not limited to, application of filtering techniques to the first detected electronic signals, application of amplification techniques to the first detected electronic signals, utilization of a lock-in amplifier on the first detected electronic signals, and so on. In some embodiments, the pre-processing may include application of a filter (e.g., bandpass or Kalman) to one or more of the detected electronic signal(s) to reduce noise or hum in the first detected electronic signals that may be caused by, for example, electronic noise generated by equipment generating and/or detecting the first detected electronic signals and/or environmental equipment that may, in some instances, be coupled to the pregnant mammal.

Optionally, in step 715, an indication of a hemoglobin oxygen saturation level for the pregnant mammal may be received from, for example, a pulse oximetry probe like pulse oximetry probe 130, a maternal pulse oximetry probe like maternal probe 133, and/or a NIRS adult hemoglobin probe like NIRS adult hemoglobin probe 125, and/or determined using, for example, a processor executing process 700 using, for example, the initial detected electronic signals. Additionally, or alternatively, an indication of a tissue oxygen saturation level for the pregnant mammal may be received and/or determined in step 715. The pregnant mammal's tissue oxygen saturation level may be received from, for example, a diffuse optical tomography (DOT) instrument and/or may be determined by applying DOT to the initial detected electronic signals. Additionally, or alternatively, an indication of a hemoglobin and/or tissue oxygen saturation level for the pregnant mammal may be determined using one or more of the first detected electronic signals received in step 705 and, for example, the Beer-Lambert Law as discussed above with regard to Equation 1 above.

In some instances, the pregnant mammal's a hemoglobin and/or tissue oxygen saturation level may be used to determine how much light is incident on the fetus as discussed below.

In step 720, a fetal depth may be received from, for example, Doppler/ultrasound probe 135 and/or may be determined using, for example, the first detected electronic signals of step 705, first fetal signals of step 710, and/or maternal hemoglobin and/or tissue saturation level of step 715.

When fetal depth is determined in step 720, the depth of the fetus may be determined by comparing an intensity of the initial fetal signals to one another to determine a change in intensity for each of the initial fetal signals. In some cases, this comparison may incorporate a position and/or source/detector distance for each detector providing a corresponding first detected electronic signal and a depth of a fetus may be determined by quantifying a drop-off, or decrease in intensity of the fetal signal, as the source/detector distance increases for detectors positioned further away from the source. This decrease in intensity as a function of source/detector distance may be used to determine a depth of the fetus.

FIG. 7B provides a flowchart illustrating an exemplary process for executing step 720 to determine a fetal depth. Initially, in step 750, a first initial detected electronic signal may be analyzed to determine if it includes an initial fetal signal. The first initial detected electronic signal may correspond to an optical signal like first optical signal 420A detected by a first detector like first detector 160A. The analysis of step 750 may be based on the processing/isolation of step 710 and may include a yes or no determination of whether or not there is any initial fetal signal to isolate from the first initial detected electronic signal. When there is an initial fetal signal included in the first initial detected electronic signal, an intensity of the initial fetal signal may be determined (step 755).

Whether or not there is an initial fetal signal included in the first initial detected electronic signal, in step 760 it may be determined if a second initial detected electronic signal includes an initial fetal signal. The second initial detected electronic signal may correspond to an optical signal like second optical signal 420B detected by a second detector like second detector 160B. Step 760 may be executed in a manner similar to the execution of step 750. When there is an initial fetal signal included in the second initial detected electronic signal, an intensity of the initial fetal signal may be determined (step 755).

Whether or not there is an initial fetal signal included in the second initial detected electronic signal, in step 765 it may be determined if a third initial detected electronic signal includes an initial fetal signal. The third initial detected electronic signal may correspond to an optical signal like third optical signal 420C detected by a third detector like third detector 160C. Step 765 may be executed in a manner similar to the execution of step 750 and/or760. When there is an initial fetal signal included in the third initial detected electronic signal, an intensity of the initial fetal signal may be determined (step 755).

A process like step 765 may be repeated N times until it is determined whether the last of the initial detected electronic signal of the plurality of initial detected electronic signal received in step 705 includes an initial fetal signal (step 770). The Nth initial detected electronic signal may correspond to an optical signal like sixth optical signal 420F detected by a sixth detector like third detector 160F. Step 770 may be executed in a manner similar to the execution of step 750,760, and/or 765. When there is an initial fetal signal included in the Nth initial detected electronic signal, an intensity of the initial fetal signal may be determined (step 755).

In step 775 a fetal depth may be determined by analyzing the determined intensities of each of the respective initial fetal signals determined in step 755. In some embodiments, step 775 may be executed by plotting (using, for example, a scatter plot) the intensities on a graph showing fetal signal intensity as a function of the detector detecting the respective 1 st-Nth fetal signal and/or as a function of a distance between a light source generating the optical signal and the detector detecting the respective 1 st-Nth initial detected electronic signals. FIG. 7C provides an example of a graph 702 showing a scatter plot of optical intensity of 1 st-Nth fetal signals in Watts/cm as a function of source/detector distance in cm that may be determined by execution of step 755. In the case of FIG. 7C, graph 702 corresponds to initial detected electronic signals detected by first through sixth detectors 160A-160F and their respective distance from light source 105. As may be seen in graph 702, a change in percent transmission of light for a signal, which may sometimes be related to the intensity of the signal, detected a distance away from the light source predominantly follows an inverse proportionality. The change in percent transmission of light and/or intensity of light incident on the fetus may vary in a nonlinear manner dependent on the fetal depth and the source-detector distance as shown graph 702 of FIG. 7C. In some embodiments, an intensity and/or change in percent transmission of light of light reaches the fetus may be dependent upon a fetal depth according to an inverse proportionality as shown in FIG. 7D, which includes a graph 703 that shows a change in percent transmission of light as a function of source-detector distance measured in cm for a maternal signal and/or a maternal portion of the signal.

Because the first fetal signal is detected by third detector with a source/detector distance of 3 cm, an exemplary depth of the fetus may be approximately 25 mm. A fetal depth may be determined using the rate of decay of the intensity of the fetal signal as the source/detector distance increases. This rate of decay may correspond to a slope of a linear regression the scatter plot.

In some embodiments, a depth of a fetus may be determined by analyzing light at the isosbestic point of 808 nm. Light at this wavelength is reflected from oxygenated and de-oxygenated hemoglobin in the same way and, as such, scattering and absorption of the light at this wavelength will be the same for both oxygenated and de-oxygenated hemoglobin and will not change based on, for example, a hemoglobin oxygen saturation level of the pregnant mammal's and/or fetus's blood. Thus, determinations of scattering and/or absorption (which may be expressed as a scattering and or absorption coefficient, respectively) of the optical signal by maternal tissue may not be required when executing step 720 using light at the isosbestic point of 808 nm.

In step 725, a second detected electronic signal that corresponds to a second optical signal may be received. The second detected electronic signal may correspond to a second optical signal that exits the abdomen of the pregnant mammal and may resemble the first detected electronic signal received in step 705. The second detected electronic signal may then be processed to isolate a portion thereof that was incident on the fetus (step 730). The isolated portion of the second detected electronic signal may be referred to herein as a second fetal signal. In some embodiments, execution of step 730 may resemble execution of step 710.

In step 735, a factor for analyzing the second fetal signal to determine a fetal hemoglobin oxygen saturation level may be selected using the fetal depth. For example, the fetal depth may be used to determine and/or select a differential path length factor (DFP) for a particular wavelength and/or a correlation factor for use in calculations to determine a hemoglobin oxygen saturation level for the fetus (step 740) using, for example, the modified Beer-Lambert law, which is presented as Equation 1 above, for each wavelength.

The fetal depth may then be used to determine and/or select the DFP for a particular wavelength. A value for lo for each wavelength of light in the incident fetal optical signal may be, for example, an intensity of light projected into the pregnant mammal's abdomen and/or an intensity of the light incident on the fetus as may be determined via a process disclosed herein. In embodiments where a hemoglobin and/or tissue oxygen saturation level of the pregnant mammal is received and/or determined in step 715, the hemoglobin and/or tissue oxygen saturation level may be used to determine how much, or an intensity of, light emitted by a light source that is directed into the abdomen of the pregnant mammal is absorbed by maternal tissue or hemoglobin. A correlation between the hemoglobin and/or tissue oxygen saturation level of the pregnant mammal and how much of the incident light she may absorb for each wavelength of light may be known and/or empirically determined and these correlations may be stored in, for example, a look up table of a database like database 170 such that when a hemoglobin and/or tissue oxygen saturation level for a pregnant mammal is received and/or determined in step 715, it may be used to look up a corresponding level of light absorption (e.g., a percentage or ratio) for the pregnant mammal. This value (the level of light absorption for the pregnant mammal) may then be applied (e.g., subtracted or multiplied) to an initial intensity of a light source when it is projecting light into the pregnant mammal's abdomen to determine the initial intensity of light incident on the fetus (I₀). Δl (λ) may be the change in the measured intensity of light incident on the fetus (I₀) at wavelength λ and an intensity of a detected fetal signal for light of wavelength λ.

Once the absorption coefficient (or a change in the absorption coefficient) is determined via Equation 1, an indication of fetal hemoglobin oxygen saturation may be determined via, for example, calculations using Equation 2, provided below:

Δμa(λ)=ΔcHbO*εHbO(λ)+ΔcHb*εHb(λ) Equation 2

where:
Δμₐ(λ)=the change in the absorption coefficient for a given wavelength λ over a defined time period;
Δc_{Hbo}=a change in the concentration of oxygenated hemoglobin (HbO) over the defined time period;
Δc_{Hb}=a change in the concentration of deoxygenated hemoglobin (Hb) over the defined time period;
ε_{Hbo}(λ)=the extinction coefficient for oxygenated hemoglobin (HbO) for the given wavelength; and
ε_{Hb}(λ)=the extinction coefficient for deoxygenated hemoglobin (Hb) for the given wavelength.

Equation 1 may be solved for two or more wavelength pairs by inputting the change in intensity I, as a function of wavelength λ. From this, changes in absorption coefficients, Δμₐ, may be determined using Equation 2 by inputting known extinction coefficients, εHbO(λ) and εHb (λ) for a particular wavelength, which may be looked up in, for example, a look-up table stored on, for example, computer 150. The wavelength pairs used to perform the calculations of Equation 2 may be any pair of wavelengths included in the spectrum of wavelengths of the optical signal incident upon the pregnant mammal's abdomen. In some embodiments, the calculation of Equation 2 may be performed many times (e.g., 10 s, 100 s, or 1000 s), in different combinations of wavelengths, in order to arrive at multiple values for ΔcHbO and ΔcHb which may be weighted and/or averaged according to one or more criteria to arrive at robust values (e.g., statistically valid and/or with an acceptable level of confidence and error rate) for ΔcHbO and ΔcHb. Additionally, or alternatively, the calculation of Equation 2 may be performed many times (e.g., 10 s, 100 s, or 1000 s), to fit a plurality of wavelengths at the same time to the equation.

The values for ΔcHbO and ΔcHb generated via Equation 2 are relative values, not absolute values, for the concentrations of oxygenated and deoxygenated hemoglobin in the fetus's blood, which may be useful in monitoring changes in the fetal hemoglobin oxygen saturation levels of the fetus over time. In some embodiments, the determination of step 1235 may also include determining an overall oxygen saturation for the fetus's hemoglobin by determining a ratio of the change in concentration of oxygenated hemoglobin to the change in concentration of total hemoglobin, which may be the sum of oxygenated and deoxygenated hemoglobin. Additionally, or alternatively, the fetal hemoglobin oxygen saturation may be determined using another method, which may be disclosed herein.

Once the fetal hemoglobin oxygen saturation level is determined in step 740, provision of an indication of same to a user may be facilitated by, for example, display on a display device like display device 155 (step 745).

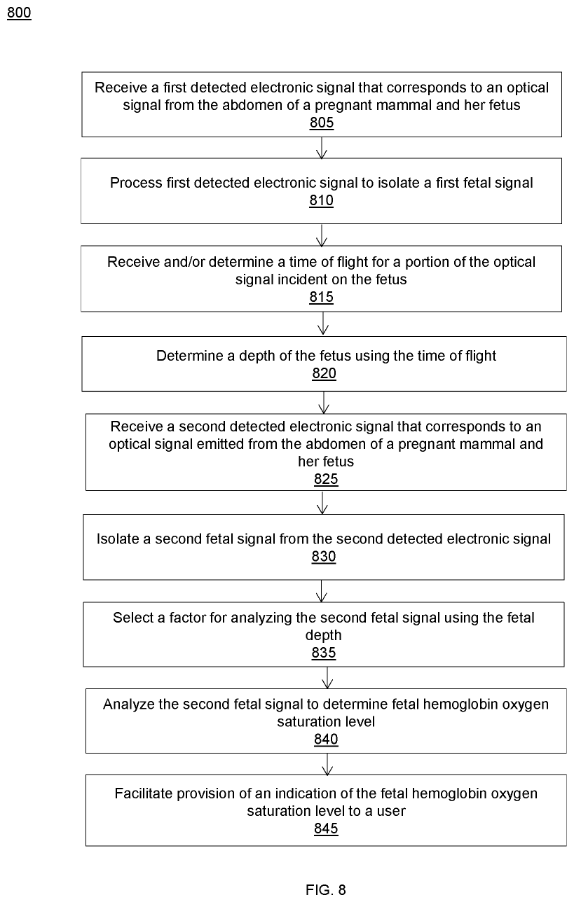

FIG. 8 is a flowchart illustrating a process 800 for determining a fetal depth using a time of flight for photons incident on a fetus and/or a level of oxygen saturation for fetal hemoglobin. Process 800 may be performed by, for example, system 100 and/or components thereof.

Optionally, in step 805, a plurality of first detected electronic signals, each of which correspond to an optical signal of one or more wavelengths projected into the pregnant mammal's abdomen by, for example, one or more light sources like light source 105 and exiting therefrom via, for example, reflection, back scattering, and/or transmission (i.e., passing through the maternal abdomen) may be received by, for example, a computer or processor such as computer 150. The first detected electronic signals received in step 805 may resemble the first detected electronic signals received in step 705. In some embodiments, a time between when the optical signal is projected into the pregnant mammal's abdomen and when it is received by a detector may be received in step 805. Additionally, or alternatively, the plurality of first detected electronic signals may include timestamp information corresponding to when, for example, the optical signal projected into the pregnant mammal's abdomen and/or when the first detected electronic signal is received by a respective detector.

When step 805 is executed, each of the first detected electronic signals received in step 805 may be processed to isolate a portion thereof that corresponds to light that has been incident upon the fetus (step 810). Execution of step 810 may resemble execution of step 710.

In step 815, an indication of a time of flight for photons of the optical signal that are incident on the fetus may be received or, when steps 805 and 810 are performed, determined using a time between when photons of the optical signal leave the light source and are received by a detector. The time of flight may be determined by calculating a length of time between when the optical signal is projected into the pregnant mammal's abdomen and when it is received by a detector. When the plurality of first detected electronic signals include timestamp information corresponding to when the optical signal projected into the pregnant mammal's abdomen and when the first detected electronic signal is received by a respective detector, a determination of the time of flight for photons of the optical signal that are incident on the fetus may be made by determining a difference, or length of time, between the timestamp for when the optical signal projected into the pregnant mammal's abdomen and the timestamp for when the first detected electronic signal is received by a respective detector.

In step 820, a fetal depth may be received from, for example, Doppler/ultrasound probe 135 and/or may be determined using the, for example, the first detected electronic signals of step 805, first fetal signals of step 810, and/or the time of flight of step 815. When fetal depth is determined in step 820, a depth of the fetus may be determined by calculating a distance traveled by the optical signal in the time between when the optical signal is projected into the pregnant mammal's abdomen and when it is received at the detector according to Equation 3, below.

D=s*t Equation 3

### Where:

D=distance traveled;

s=speed of light; and

t=time between when the optical signal is projected into the pregnant

mammal's abdomen and when it is received at the detector

The fetal distance may be calculated by dividing a value for the distance traveled by the light (D) by 2 because the distance calculated via Equation 3 is the distance the light travels to the fetus and back to the detector.

In step 825, a second detected electronic signal that corresponds to a second optical signal may be received. The second detected electronic signal may correspond to a second optical signal that exits the abdomen of the pregnant mammal and may resemble the first detected electronic signal received in step 805. The second detected electronic signal may then be processed to isolate a portion thereof that was incident on the fetus (step 830). The isolated portion of the second detected electronic signal may be referred to herein as a second fetal signal. In some embodiments, execution of step 830 may resemble execution of step 810 and/or step 730.

In step 835, a factor for analyzing the second detected electronic signal to determine a fetal hemoglobin oxygen saturation level may be selected using the fetal depth. For example, the fetal depth may be used to determine and/or select a differential path length factor (DFP) for a particular wavelength and/or a correlation factor for use in calculations to determine a hemoglobin oxygen saturation level for the fetus (step 840) using, for example, Equations 1 and 2, provided and discussed above. In some embodiments, execution of step 835 may resemble execution of step 735. Additionally, or alternatively, the fetal hemoglobin oxygen saturation may be determined using another method, which may be disclosed herein.

Once the fetal hemoglobin oxygen saturation level is determined in step 840, provision of an indication of same to a user may be facilitated by, for example, display on a display device like display device 155 (step 845).

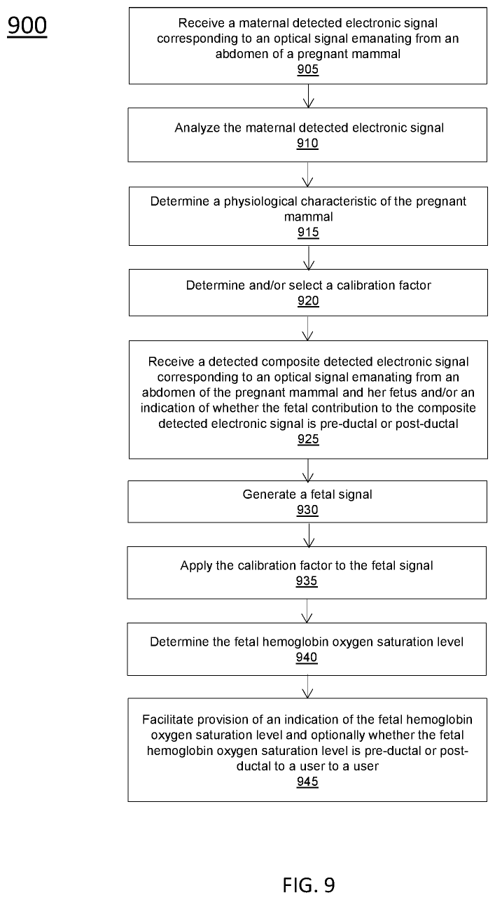

FIG. 9 provides a flowchart illustrating a process 900 for determining a fetal hemoglobin oxygenation saturation level using physiological characteristics of the pregnant mammal determined using a maternal detected electronic signal. Process 900 may be performed by, for example, system 100 and/or components thereof. Process 900 may be executed in-situ during, for example, a labor and delivery of the fetus and/or a wellness checkup for the pregnant mammal. In some cases, process 900 may be executed on a continuous, periodic, and/or as-needed basis over a period of time such as the labor and delivery of the fetus so that, for example, the fetal hemoglobin oxygenation saturation level may be calibrated and recalibrated over time as needed and/or when conditions for the fetus and/or pregnant mammal change as may be the case when, for example, the fetus travels through the birth canal.

In step 925, a detected composite electronic signal may be received from the detector by a processor and/or computer like computer 150. The detected composite electronic signal may be received from, for example, a photo-detector, a transceiver coupled to the photo-detector, and/or a fetal hemoglobin probe such as fetal hemoglobin probe 115. The detected composite electronic signal may correspond to an optical signal emanating from the abdomen of a pregnant mammal and/or her fetus. Light incident upon, and exiting from, the pregnant mammal's abdomen may be generated by one or more light sources like light sources 105 and may be of any acceptable frequency or wavelength (e.g., near infra-red (NIR)) and/or combination of frequencies and/or wavelengths. In some embodiments, an indication of whether the fetal hemoglobin oxygen saturation level is pre-ductal or post-ductal may also be received in step 925 in a manner similar to, for example, the receipt of an indication of whether the fetal hemoglobin oxygen saturation level is pre-ductal or post-ductal in step 1305. Step 925 may be executed at any point following execution of step 920. However, in many cases, step 925 may be performed immediately, or soon (e.g., 5 seconds, 30 seconds, 1 minute) following execution of step 920 to, for example, account for dynamic changes to the physiological characteristics of the pregnant mammal's abdomen.

Next, the received detected composite electronic signal may be analyzed to isolate a portion of the signal that has corresponds to light that was incident upon the fetus, thereby generating a fetal signal (step 930). Step 930 may be executed in a manner similar to the execution of, for example, step 810 and/or 710 or any other method disclosed herein. Then, the calibration factor selected and/or determined in step 920 may be applied to the fetal signal in order to calibrate the fetal signal (step 935). Once the fetal signal has been calibrated, an indication of the fetal hemoglobin oxygen saturation level may be determined (step 940) and provided to a user (step 945). Optionally, execution of step 945 may include an indication of whether the fetal hemoglobin oxygen saturation level is pre-ductal or post-ductal to the user in manner similar to the execution of step 1355. Execution of steps 935-945 may be similar to execution of steps 1345-1350 disclosed below and/or steps 830-835. Additionally, or alternatively, the fetal hemoglobin oxygen saturation may be determined using another method, which may be disclosed herein.

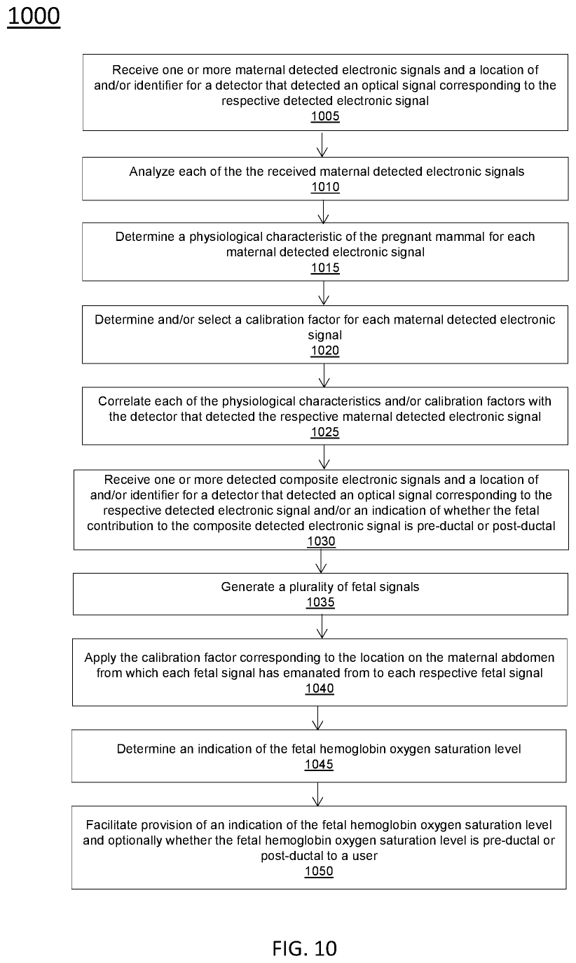

FIG. 10 provides a flowchart illustrating a process 1000 for determining a fetal hemoglobin oxygenation saturation level using physiological characteristics of the pregnant mammal determined using one or more maternal detected electronic signal(s). Process 1000 may be used to account for changes in maternal geometries and/or geometrical physiological characteristics across, for example, the surface area of a fetal hemoglobin probe like fetal hemoglobin probe 115. Process 1000 may be performed by, for example, system 100 and/or components thereof. Process 1000 may be executed in-situ during, for example, a labor and delivery of the fetus and/or a wellness checkup for the pregnant mammal. In some cases, process 1000 may be executed on a continuous, periodic, and/or as-needed basis over a period of time (e.g., 1-24 hours) such as the labor and delivery of the fetus so that, for example, the fetal hemoglobin oxygenation saturation level may be calibrated and recalibrated over time as needed and/or when conditions for the fetus and/or pregnant mammal change as may be the case when, for example, the fetus travels through the birth canal.

Initially, in step 1005, one or more maternal detected electronic signal(s) corresponding to one or more optical signal(s) emanating from the abdomen of a pregnant mammal may be received (step 1005). The optical signal may be generated by a light source like light source 105, incident on the pregnant mammal's abdomen, pass through a portion of the maternal abdominal tissue, and be reflected, or backscattered, through the maternal tissue where it is detected by a detector like detector 160. Each optical signal may be associated with a location of and/or identifier for each detector that detected the respective optical signal. For example, each detector that detects a maternal detected electronic signal may be associated with an identifier (e.g., detector 1, detector 2, etc.), a location that may be a location (e.g., coordinates) of the detector on a fetal hemoglobin probe 115 and/or a location on the maternal abdomen (e.g., 1 inch directly below the navel, 1 inch below the navel and 1 inch to the left of the midsagittal line, etc.). Exemplary optical signals like the optical signal that may be detected by a detector and received in step 1005 are shown in, for example, FIG. 3C as first and second optical signals 420A and 420B and are shown as optical signals 420 in FIGS. 4A-4D.

In step 1010, each of the maternal detected electronic signals may be analyzed to determine one or more extrinsic or geometrical physiological characteristics of the pregnant mammal (step 1015). Exemplary extrinsic physiological characteristics include, but are not limited to, melanin content and/or degree and/or type of skin pigmentation of pregnant mammal's skin, a width of one or more layers of maternal or fetal tissue, abdominal wall thickness for the pregnant mammal. Exemplary dimensions for the thickness of an abdominal wall and/or layers of the pregnant mammal's abdominal wall are provided above with regard to FIGS. 3A and 3B.

In step 1020, a calibration factor may be selected and/or determined for each maternal detected electronic signal. The determination (i.e., calculation) and/or selection of a calibration factor may be made by, for example, assessing how much light from the incident optical signal is absorbed and/or scattered by the pregnant mammal's abdominal tissue and/or a time of flight for photons of the optical signal to travel through the pregnant mammal's abdominal tissue and be detected by the detector. In some embodiments, the selection of a calibration factor may be performed by querying a database like database 170 and/or a memory resident within a computer such as computer 150 for calibration factors that correspond to the physiological characteristic determined in step 915. The database may be populated with correlated physiological characteristics and calibration factors via process 500. In some embodiments, the results of executing steps 1005, 1010, 1015, and/or 1020 may be stored in, for example, a database like database 170 and/or a memory resident within a computer such as computer 150.

In step 1025, the physiological characteristics and/or calibration factors may be associated with the detector that detected each of the respective maternal detected electronic signals received in step 1005. Additionally, or alternatively, in step 1025, the physiological characteristics and/or calibration factors may be associated with a location on the pregnant mammal's abdomen.

In one example, a set of results from executing steps 1005-1025 is provided by the values in Table 1, below which provides a detector identifier, a value for the physiological characteristic of abdominal thickness, and a calibration factor corresponding to the abdominal thickness physiological characteristic.

TABLE 1

| Detector Identifier | Abdominal Thickness | Calibration Factor |
|---|---|---|
| Detector 1 | 19.81 mm | C1 |
| Detector 2 | 19.83 mm | C2 |
| Detector 3 | 19.85 mm | C3 |

In step 1030, one or more detected composite electronic signal(s) may be received by a processor and/or computer like computer 150. The one or more detected composite electronic signal(s) may be received from the detector(s) that provided the maternal detected electronic signals received in step 1005 and each of the detected composite electronic signal(s) may be associated with a location of and/or identifier for the detector that detected each respective detected composite electronic signal(s). In some embodiments, an indication of whether the fetal hemoglobin oxygen saturation level is pre-ductal or post-ductal may also be received in step 1030 in a manner similar to, for example, the receipt of an indication of whether the fetal hemoglobin oxygen saturation level is pre-ductal or post-ductal in step 1305.

The detected composite electronic signal may be received from, for example, a photo-detector, a transceiver coupled to the photo-detector, and/or a fetal hemoglobin probe such as fetal hemoglobin probe 115. The detected composite electronic signal may correspond to an optical signal emanating from the abdomen of a pregnant mammal and/or her fetus. Light incident upon, and exiting from, the pregnant mammal's abdomen may be generated by one or more light sources like light sources 105 and may be of any acceptable frequency or wavelength (e.g., near infra-red (NIR)) and/or combination of frequencies and/or wavelengths. Step 1030 may be executed at any point following execution of step 1025. However, in many cases, step 1030 may be performed immediately, or soon (e.g., 5 seconds, 30 seconds, 1 minute, 1 hour) following execution of step 1025 to, for example, account for dynamic changes to the physiological characteristics of the pregnant mammal's abdomen.

Next, the received detected composite electronic signal(s) may be analyzed to isolate a portion of the respective composite electronic signal that has corresponds to light that was incident upon the fetus, thereby generating a corresponding number of fetal signal(s) (step 1035). Step 1035 may be executed in a manner similar to the execution of step(s) 710 and/or 810 and/or in accordance with other method(s) disclosed herein. Then, the calibration factor correlated with the detector that detected each respective detected composite electronic signal may be applied to the fetal signal corresponding to each respective detected composite electronic signal (step 1040). Execution of step 1040 may resemble execution of step 935 as discussed above.

Continuing the example above, application of the calibration factors to fetal signals received from the first, second, and third detectors in step 1040 corresponds to first, second, and third fetal signals (i.e., fetal signal 1, fetal signal 2, and fetal signal 3), which corresponds to calibration factors P1, P2, and P3, respectively as shown in Table 2, below where the detector the calibration factor of a first, second, and third physiological characteristic corresponds to the calibration factor for the first, second, and third fetal signal, respectively, as shown in Table 2.

TABLE 2

| Detector Identifier | Fetal Signal Identifier | Calibration Factor |
|---|---|---|
| Detector 1 | Fetal Signal 1 | P1 |
| Detector 2 | Fetal Signal 2 | P2 |
| Detector 3 | Fetal Signal 2 | P3 |

In some embodiments, the fetal signals disclosed herein and/or fetal signal 1, fetal signal 2, and/or fetal signal 3 may be a signal that includes light of a plurality of wavelengths and a calibration factor for each of these wavelengths may be separately determined and/or applied to the individual wavelengths (or groups of similar wavelengths) included in each fetal signal. Additionally, or alternatively, the fetal signals disclosed herein and/or fetal signal 1, fetal signal 2, and/or fetal signal 3 may each be detected by a separate detector and/or be detected at a different time by the same detector. For example, a fetal pulse may be extracted from a plurality fetal signals (each of which may be detected by a separate detector) and the calibration(s) of the fetal signals may be influenced differently in each detector channel by different proportionalities of the different physical characteristics, which may be governed by the following relationship: if detector 1 signal =function D1 (D1, P1, P2, P3, . . . ) where D1 is a vector of coefficients associated with the detector 1 geometry, wavelength, etc. and P1 is a vector associated with physical characteristics P1, P2 is a vector associated with physical characteristics P2, and so forth. In this example, the function D1 may be considered as a tensor.

Once each of the fetal signals have been calibrated, an indication of the fetal hemoglobin oxygen saturation level may be determined (step 1045) using, for example, one or more of the methods disclosed herein. In some cases, step 1050 may be executed by individually determining a fetal hemoglobin oxygen saturation level for each fetal signal and then averaging the individually determined fetal hemoglobin oxygen saturation level into an average a fetal hemoglobin oxygen saturation level. Additionally, or alternatively, each fetal signal may be a mix, or combination, of different signals coming from the various simultaneously occurring physical characteristics and, in some cases, each calibration factor C1, C2, etc., may be a vector and for the family of detector signals, it would be a tensor matrix.

Once the fetal hemoglobin oxygen saturation level is determined, the fetal hemoglobin oxygen saturation level may be provided to a user (step 1050). Optionally, execution of step 1050 may include an indication of whether the fetal hemoglobin oxygen saturation level is pre-ductal or post-ductal to the user in manner similar to the execution of step 1355. In some embodiments, execution of steps 1045 and 1050 may be similar to execution of steps p 1345-p 1350, steps 830-835, and/or steps 940 and 945.

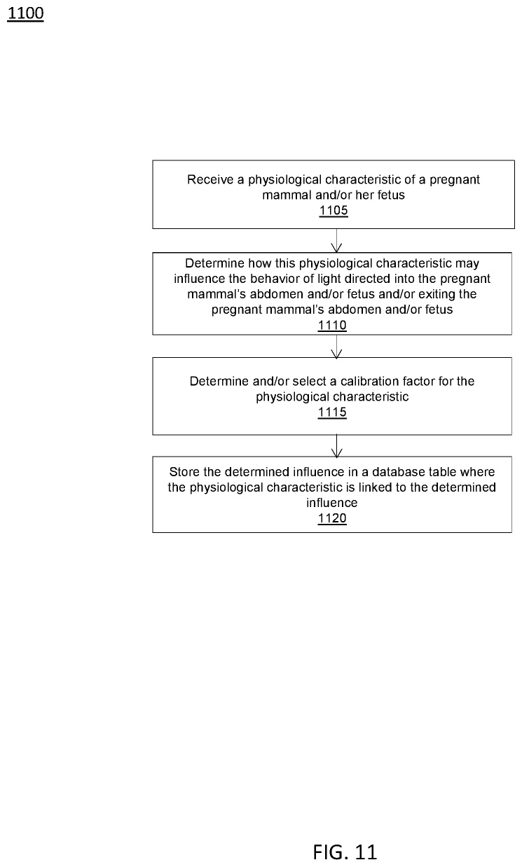

FIG. 11 provides a flowchart illustrating a process 1100 for determining an influence of a physiological characteristic on the behavior of light (e.g., scattering, absorption, etc.) traversing through the abdomen of a pregnant mammal and/or her fetus. Process 1100 may be executed by, for example, system 100 and/or a component combination of components thereof. Exemplary physiological characteristics include, but are not limited to, type of tissue, a depth of tissue, a width of a layer of tissue, how many layers of tissue the light is illuminating, skin pigmentation, density of tissue or a layer of tissue, a depth of the fetus within the maternal abdomen, composition of tissue or a layer of tissue, and so on.

In step 1105, a physiological characteristic of a pregnant mammal and/or her fetus may be received. In some cases, the physiological characteristic may be determined via, for example, analyzing an image of the pregnant mammal and/or her fetus. The image may be generated by one or more imaging techniques including, but not limited to, MRI or ultrasound imaging technologies. Illustrations of exemplary images that may be analyzed to determine one or more physiological characteristics include those of FIGS. 3A and 3B. Additionally, or alternatively, the physiological characteristic may not be based upon an image, such as body mass index, skin color, age, and so on.

In step 1110, it may be determined how the physiological characteristic may influence the behavior of light directed into and/or passing through the maternal abdomen and/or fetus. The light directed into and/or passing through the maternal abdomen and/or fetus may be of a single or multiple (e.g., a broad or narrow range) wavelength(s) and the determination may be based upon and/or factor in one or more wavelengths of interest. In general, light's behavior (e.g., scattering) is dependent upon tissue morphology and particle size/density for matter (e.g., water, lipids, etc.) within tissue. Different layers of tissue typically have different morphology, particle size and/or particle density. For example, when a physical characteristic of a layer of tissue is a higher than average amount of fat (i.e., high lipid count) within the tissue, then one might expect the amount of scattering of the light to be higher than average due to the higher than average lipid content. In some embodiments, this influence may be described by a scattering coefficient (e.g., ps(A)), an absorption coefficient (e.g., μₐ(λ)), an adjustment to a scattering coefficient, and/or an adjustment to an absorption coefficient that may be input into an equation pertaining to, and/or incorporating, the behavior of light as it passes through a medium or a plurality of mediums such as the Beer-Lambert Law and/or Modified Beer-Lambert Law.

The determination of step 1110 may be done using, for example, experimental observations gathered from one or more pregnant mammals and/or mathematical modeling performed for a plurality of theoretical pregnant mammals and/or theoretically modeled physiological characteristics. For example, experimental observations may be used to correlate observed optical signal behavior that may be detected upon exiting from the abdomen of a pregnant mammal under study (via, for example, reflection, back scattering, and/or transmission) and physiological characteristics of the pregnant mammal/pregnant mammal's abdomen. Additionally, or alternatively, detected light scattering and/or detected optical signals for a cohort of pregnant mammals may be analyzed to determine how various physiological characteristics of the pregnant mammals in the cohort may impact light scattering. For instance, light scattering and/or detected optical signals may be analyzed along with physiological characteristics such as a width of a subcutaneous fat layer for each pregnant mammal of the cohort to determine whether the width of the subcutaneous fat layer impacts the behavior of the detected light and, if so, how the light's behavior is impacted and to what degree. This process may be repeated with, for example, a plurality of physiological characteristics of the pregnant mammals within the cohort, a plurality of tissue layers of the pregnant mammal's abdomen for each pregnant mammal of the cohort in isolation (i.e., one at a time) and/or in combination (i.e., determine how a plurality of physiological characteristics may impact the behavior of light). In some embodiments, the determinations of step 1110 may be dependent on the frequency/wavelength of light being observed or measured. In some cases, the determinations of step 1110 may consider how different intrinsic and/or tissue properties and geometric properties of the pregnant mammal and/or fetus may impact different wavelengths. For instance, a particular physiological characteristic may impact the behavior of light of a first wavelength (e.g., 700 nm) differently than it impacts light of a second wavelength (e.g., 800 nm) by, for example, having greater/lesser scattering and/or absorption of the light of a first wavelength when compared with light of a second wavelength. Thus, the impact of a physiological characteristic may be determined for different frequencies/wavelengths and/or different ranges of frequencies/wavelengths of light.

Additionally, or alternatively, when step 1110 is executed using mathematical modeling, one or more known, understood, estimated, and/or assumed ways light may behave when passing through a material (e.g., skin, water, water with a known lipid count, skeletal muscle, fat, smooth muscle, water with a known electrolyte count, etc.) may be used to mathematically model how light may behave when passing through the material, and/or a plurality of materials (e.g., skin, fat, muscle, etc.) in a physiological context, such as through a pregnant mammal's abdomen. Exemplary programs that may be used to perform the mathematical modeling include, but are not limited to, Monte Carlo simulations and NIRFAST for finite element modeling.

In one example, the physiological characteristic received in step 1105 may be a skin color, pigmentation and/or melanin content that is input by, for example, a clinician or doctor. In some embodiments, a skin color may be quantified using the Fitzpatrick scale and may be input into a processor or computer executing process 1100 by the clinician. In step 1110, it may be determined how the quantified skin color of the pregnant mammal may influence the behavior of light incident on the pregnant mammal's abdomen and/or emanating from her skin. The skin color of the pregnant mammal's skin may influence how much of the incident light is absorbed, which may impact how much of the light incident on the pregnant mammal's abdomen travels through the maternal tissue and is incident on the fetus according to a known, or calculated, factor, which is determined in step 1110.

In another example, the physiological characteristic received in step 1105 may be a density, concentration, and/or thickness of the myoglobin (which may be collectively referred to herein as myoglobin concentration), or muscle, layers of the pregnant mammal's abdomen. The myoglobin tissue of the pregnant mammal may absorb light projected into the maternal abdomen and a measurement of how concentrated a myoglobin layer is may be used to determine how much light is absorbed (e.g., not detected) by the pregnant mammal's myoglobin tissue. In step 620, this physiological characteristic may be used to, for example calculate a calibration factor and/or query a database of calibration factors to find a calibration factor associated with the physiological characteristic of the myoglobin concentration determined for the pregnant mammal.

In another example, the physiological characteristic received in step 1105 may be a total thickness of pregnant mammal's abdomen (which may also be referred to herein as fetal depth) which may vary over the course of a pregnancy due to the increasing size of the fetus. In some cases, the thickness of the pregnant mammal's abdomen may be determined via, for example, a body mass index (BMI) calculation, analysis of an image, exemplary illustrations of which are shown in illustrations 301 and 302 and/or analysis of an ultrasound image. Additionally, or alternatively, the thickness of the pregnant mammal's abdominal tissue may vary of the course of the pregnancy and/or during labor and delivery of the fetus because of pre-eclampsia or eclampsia. The abdominal tissue of the pregnant mammal may absorb light projected into the maternal abdomen and a measurement and/or calculation of how much light that is absorbed (e.g., not detected) by the pregnant mammal's abdominal tissue may be used to determine the physiological characteristic of how thick the pregnant mammal's abdominal tissue is. In step 1115, this physiological characteristic may be used to, for example determine a calibration factor and/or query a database of calibration factors to find a calibration factor associated with the physiological characteristic of the abdominal thickness determined for the pregnant mammal.

In yet another example, the physiological characteristic received in step 1105 may be a thickness of the adipose, or fat, layers of the pregnant mammal's abdomen. The adipose tissue of the pregnant mammal may scatter light projected into the maternal abdomen and a measurement and/or calculation of how thick an adipose layer is may be used to determine how much light the adipose layer may scatter. In step 1115, this physiological characteristic may be used to, for example calculate a calibration factor and/or query a database of calibration factors to find a calibration factor associated with the physiological characteristic of the adipose thickness determined for the pregnant mammal.

In a different example, the physiological characteristic received in step 1105 may be an amount of hemoglobin circulating in the pregnant mammal's blood (i.e., hemoglobin concentration). An amount of hemoglobin circulating in the blood of a pregnant mammal may be determined via a blood measurement such a hemoglobin concentration measurement, a hematocrit measurement, and/or total blood volume measurement to quantify this the maternal hemoglobin concentration. In some cases, a measurement of hemoglobin concentration may be measured using a device like the Masimo's SpHb device which is configure to non-invasively measure hemoglobin concentration.

Anemia is a condition that causes a decrease in hemoglobin concentrations in the pregnant mammal's blood and a condition such as polycythemia vera causes an increase in hemoglobin concentration in the pregnant mammal's blood. The hemoglobin concentration in the pregnant mammal's blood may absorb light projected into the maternal abdomen and a value for the hemoglobin concentration in the pregnant mammal's blood may be used to determine how much light the mother's hemoglobin may absorb, wherein an anemic pregnant mammal's blood would not absorb as much light as a pregnant mammal with a normal value for hemoglobin concentration, which may impact how much light is incident on the fetus. Likewise, the increased hemoglobin concentration of a pregnant mammal with polycythemia vera may absorb more light than a pregnant mammal with a normal value for hemoglobin concentration, which may impact how much light is incident on the fetus. In step 1115, this physiological characteristic may be used to, for example calculate a calibration factor and/or query a database of calibration factors to find a calibration factor associated with the physiological characteristic of the adipose thickness determined for the pregnant mammal.

In another example, the physiological characteristic received in step 1105 may be a hemoglobin oxygen saturation measurement for the pregnant mammal that may be measured by, for example, analysis of a direct arterial blood sample, approximated using a venous blood sample, a pulse oximeter like pulse oximeter 130 and/or a NIRS adult hemoglobin probe like NIRS adult hemoglobin probe. Conditions such as pneumonia, asthma, COVID-19, cardiovascular conditions that may affect blood oxygen saturation, and high altitude can all cause a drop in the maternal hemoglobin oxygen saturation. The hemoglobin oxygen saturation in the pregnant mammal's blood may determine how much light the oxygenated and/or deoxygenated hemoglobin absorbs and a value for the hemoglobin oxygen saturation of the pregnant mammal's blood may be used to determine how much light the mother's oxygenated/deoxygenated hemoglobin may absorb, which may impact how much light is incident on the fetus.

In step 1120, the determined influence(s) of the one or more physiological characteristics and/or combination(s) of physiological characteristics on light's behavior may be stored in a database, like database 170. At times, a physiological characteristic may be indexed within the database to a corresponding determination of the physiological characteristic's influence on light, or a particular wavelength of light. For example, a physiological characteristic that is static over a period of time (e.g., anemia, hypertension, respiratory illness, and/or a traditionally low blood oxygen level for the pregnant mammal) may be indexed to and/or correlated with a corresponding determination of the physiological characteristic's influence on light, or a particular wavelength of light (e.g., a calibration factor).

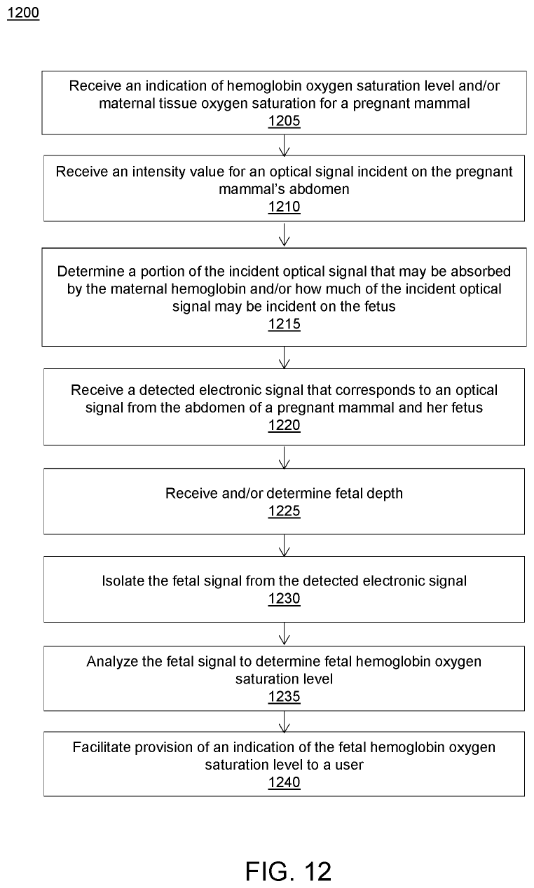

FIG. 12 provides a flowchart illustrating an exemplary process 1200 for determining a fetal hemoglobin oxygen saturation level using an indication of a maternal hemoglobin oxygen saturation level and/or a fetal depth. Process 1200 may be executed by, for example, system 100 and/or a component thereof.

In step 1205, an indication of a hemoglobin oxygen saturation level for a pregnant mammal may be received from, for example, a pulse oximetry probe like pulse oximetry probe 130, a maternal pulse oximetry probe like maternal probe 133, and/or a NIRS adult hemoglobin probe like NIRS adult hemoglobin probe 125 and/or determined using, for example, a processor executing process 1200 using, for example, the first detected electronic signals. Additionally, or alternatively, an indication of a tissue oxygen saturation level for the pregnant mammal may be received and/or determined in step 715. The pregnant mammal's tissue oxygen saturation level may be received from, for example, a diffuse optical tomography (DOT) instrument and/or may be determined by applying DOT to the first detected electronic signals. In some embodiments, execution of step 1205 may resemble execution of step 715.

In step 1210, an intensity value for an optical signal incident on the pregnant mammal's abdomen may be received. This intensity value may be known from, for example, a manufacturer of a light source being used to generate the optical signal and/or may be experimentally determined. In step 1215, a portion of the incident optical signal that may be absorbed by the pregnant mammal and therefore may not be incident on the fetus and/or how much of the incident signal may be incident on the fetus may be determined and/or received. The portion of the incident optical signal that may be incident on the fetus may be referred to herein as the incident fetal optical signal. Step 1215 may be determined by using a light absorption rate (e.g., Δμa(π)) of the pregnant mammal that may be based on her hemoglobin and/or tissue oxygenation level received in step 1205. In some embodiments, the determination of step 1215 may resemble execution of step 720.

In step 1220, a detected electronic signal may be received from a photo-detector, like detector 160. The detected electronic signal may correspond to an optical signal of one or more wavelengths incident upon, and exiting from, an abdomen of a pregnant mammal and her fetus that has been detected by the detector over a period of time. Detection of the optical signal may include counting photons of different wavelengths that are received by the detector and/or incident on optical fibers coupled to the detector. On some occasions, the received detected electronic signals may resemble the detected electronic signals received in step 705 and/or 725 of process 700 and/or steps 805 and/or 825 of process 800, discussed above.

Optionally, in some embodiments, a fetal depth (e.g., a distance between the epidermis of the pregnant mammal's abdomen and the epidermis of the fetus) may be received and/or determined (step 1225). The fetal depth may be received from, for example, a Doppler/ultrasound probe like Doppler/ultrasound probe 135 and/or a fetal depth probe like fetal depth probe 138. The fetal depth may be determined via, for example, execution of process 700 and/or 800 described above with regard to FIGS. 7 and 8, respectively. When a fetal depth is received and/or determined in step 1225, the fetal depth may be used to determine and/or select a calibration factor and/or a differential path length factor (DPF) for one or more wavelength(s) of light included in the fetal signal and/or incident optical signal.

In step 1230, a portion of the detected electronic signal of step 1220 that has been incident upon the fetus may be isolated from the detected electronic signal according to, for example, one or more methods disclosed herein. This isolated portion of the received detected electronic signal may be referred to herein as a fetal signal. Step 1230 may be executed using any appropriate method of isolating the fetal signal from the detected electronic signal. Appropriate methods include, but are not limited to, reducing noise in the signal via, for example, application of filtering or amplification techniques, determining a portion of the detected electronic signal that is contributed by the pregnant mammal and then subtracting, or otherwise removing, that portion of the detected electronic signal from the received detected electronic signals and/or receiving information regarding fetal a heart rate and using that information to lock in (via, for example, a lock-in amplifier) on a portion of the received detected electronic signals generated by the fetus.

In step 1235, the fetal signal may be analyzed to determine a fetal hemoglobin oxygen saturation level using, for example, Equations 1 and 2, described above and/or one or more of the methods disclosed herein. In some embodiments, execution of step 1235 may resemble execution of steps 740 and/or 840 of process(es) 700 and 800, respectively. Once the fetal hemoglobin oxygen saturation level is determined in step 1235, provision of an indication of same to a user may be facilitated by, for example, display on a display device like display device 155.

FIG. 13 provides a flowchart illustrating a process 1300 for determining a fetal hemoglobin oxygenation saturation level using calibration factor and/or physiological characteristic of the pregnant mammal and/or fetus. Process 1300 may be performed by, for example, system 100 and/or components thereof.

Initially, a detected composite electronic signal may be received from a photo-detector (e.g., detector 160) by a processor and/or computer like computer 150 (step 1305). The detected composite electronic signal may be received from, for example, a photo-detector, a transceiver coupled to the photo-detector, and/or a fetal hemoglobin probe such as fetal hemoglobin probe 115. The detected composite electronic signal may correspond to an optical signal emanating from the abdomen of a pregnant mammal and/or her fetus. Light incident upon, and exiting from, the pregnant mammal's abdomen may be generated by one or more light sources like light sources 105 and may be of any acceptable frequency or wavelength (e.g., near infra-red (NIR)) and/or combination of frequencies and/or wavelengths. In some embodiments, (e.g., when multiple detectors are used) the received detected composite electronic signals may be include and/or be associated with a detector identifier so that a position of a particular detected composite electronic signal may be known. This location may then be used to analyze the received detected composite electronic signals to determine various factors of the detected light and/or imaged tissue.

In some embodiments, an indication of whether the fetal hemoglobin oxygen saturation level is pre-ductal or post-ductal may also be received in step 1305. This indication may, in some cases, be an indication of where, on the fetus, the optical signal is reflecting from wherein a measurement from the head of the fetus would indicate a pre-ductal measurement (i.e., higher fetal oxygen saturation level) and measurement from the body of the fetus (e.g., back or buttocks) would indicate a post-ductal measurement were a lower fetal oxygen saturation level is expected. The indication of where on the fetus the measurement is reflecting from may be provided by, for example, a user who enters this information following examination of an image, like an ultrasound or MRI image of the pregnant mammal's abdomen.

Optionally, information regarding the fetus and/or pregnant mammal may be received (step 1310). Exemplary information includes, but is not limited to, fetal heart rate, a fetal ECG signal, a maternal heart rate, a maternal ECG signal, uterine contraction information for the pregnant mammal, maternal hemoglobin oxygen saturation, and/or a maternal respiration signal.

Next, the received detected composite electronic signal may be analyzed to isolate a portion of the signal that has corresponds to light that was incident upon the fetus, thereby generating a fetal signal (step 1315). Step 1315 may be executed using any appropriate method of isolating the fetal signal from the received detected composite electronic signal. Appropriate methods include, but are not limited to application of filtering or amplification techniques, determining a portion of the detected composite electronic signal that is contributed by the pregnant mammal and then subtracting or otherwise removing that portion of the detected composite electronic signal from the detected composite electronic signal and/or receiving information regarding fetal heart rate and using that information to lock in (via, for example, a boxcar and/or gated integrator and/or a lock-in amplifier) on a portion of the detected composite electronic signal that may be generated and/or influenced by the fetus. When information is received in step 1310, execution of step 1315 may include using the information received in step 1310 to generate the fetal signal.

In step 1320, it may be determined if the pregnant mammal is associated with a calibration factor. The pregnant mammal may be associated with a calibration factor via, for example, execution of process 500 that may be done proximate in time (e.g., contemporaneously, within a few minutes, hours, days, and/or weeks) to the receipt of the detected composite electronic signal in step 1305. In some cases, the calibration factor may be specific to the pregnant mammal and/or week of gestation for her fetus. When the pregnant mammal is associated with a calibration factor, process 1300 may proceed to step 1345, and the calibration factor may be received.

Additionally, or alternatively, one or more physiological characteristics of the pregnant mammal and/or fetus may be requested and/or determined (step 1325). A physiological characteristic may be determined via, for example, analysis of an image of the pregnant mammal's abdomen and/or a measurement of a fetal depth. Exemplary requests may take the form of, for example, provision of a question or request to a user fetal hemoglobin probe 115. The physiological characteristic may be received via any appropriate means including, but not limited to, direct entry by a user via an interface (e.g., key pad or microphone), querying a database for medical/physiological information regarding the pregnant mammal, etc. In some instances, the physiological characteristic will be demographic (e.g., age or skin tone) and/or related to the pregnancy (e.g., weeks of gestation, position of the fetus within the abdomen, etc.). In step 1330, a physiological characteristic for the pregnant mammal and/or fetus may be received. Then a database, like database 170, may be queried using the received physiological characteristic to determine and/or select one or more calibration factor(s) that is/are appropriate for the pregnant mammal and/or fetus (step 1335).

The queried-for calibration factor(s) may be received in step 1340 and applied to the fetal signal in order to, calibrate, or otherwise improve (e.g., clarify or improve accuracy of) the fetal signal and the calibrated signal may then be used to determine a level of fetal hemoglobin oxygen saturation thereby generating a calibrated fetal signal (step 1345). In some embodiments, execution of step 1345 may include using each of the calibration factors received in step 1340 to generate a corresponding calibration curve that considers one or more physiological characteristic of the pregnant mammal and/or fetus and then applying these calibration curve(s) to the fetal signal to calibrate and/or improve the fetal signal.

In step 1350, the calibrated fetal signal may be analyzed to determine the fetal hemoglobin oxygen saturation and provision of an indication of the level of fetal hemoglobin oxygen saturation to a user may be facilitated by, for example, computer 150 and/or a display device (step 1355). Optionally, execution of step 1355 may include an indication of whether the fetal hemoglobin oxygen saturation level is pre-ductal or post-ductal to the user. The indication of whether the fetal hemoglobin oxygen saturation level is pre-ductal or post-ductal may help a clinician determine whether the fetal hemoglobin oxygen saturation level is low enough to cause concern (e.g., indicate that there is a possibility of fetal acidosis) or to warrant further intervention like a Caesarian section. Further details regarding the execution of step 1340 are provided below with regard to the discussion of process 1400, and, in particular execution of steps 1425 and 1430.

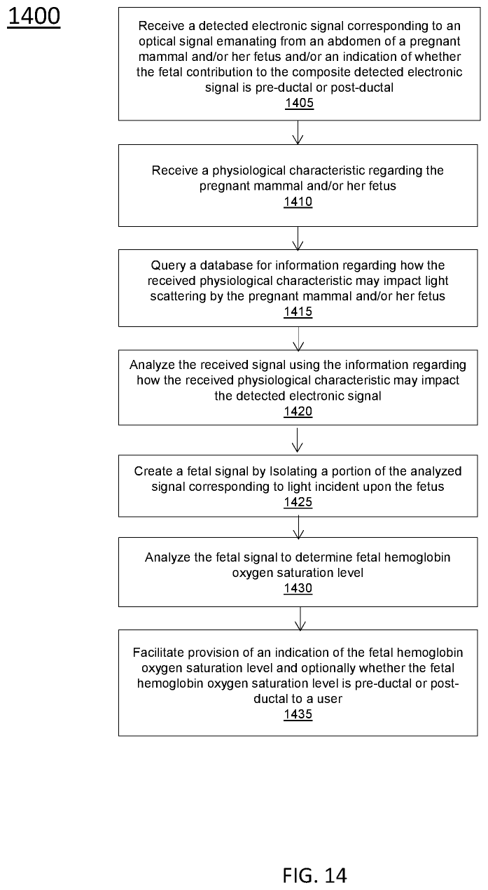

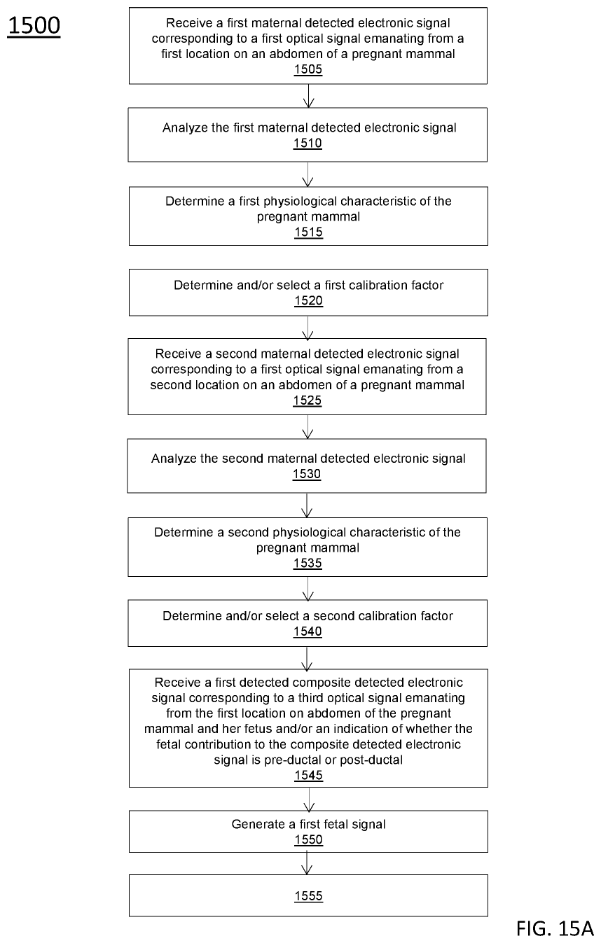

FIG. 14 provides a flowchart illustrating a process 1400 for determining a fetal hemoglobin oxygenation saturation level using physiological characteristics of the pregnant mammal and/or fetus. Process 1400 may be performed by, for example, system 100 and/or components thereof.

Initially, a detected composite electronic signal may be received from a photo-detector (e.g., detector 160) by a processor and/or computer like computer 150 (step 1405). The detected composite electronic signal may be received from, for example, a photo-detector, a transceiver coupled to the photo-detector, and/or a fetal hemoglobin probe such as fetal hemoglobin probe 115. In some embodiments, an indication of whether the fetal hemoglobin oxygen saturation level is pre-ductal or post-ductal may also be received in step 1405 in a manner similar to, for example, the receipt of an indication of whether the fetal hemoglobin oxygen saturation level is pre-ductal or post-ductal in step 1405.

The detected composite electronic signal may correspond to an optical signal emanating from the abdomen of a pregnant mammal and/or her fetus. Light incident upon, and exiting from, the pregnant mammal's abdomen may be generated by one or more light sources like light sources 105 and may be of any acceptable frequency or wavelength (e.g., near infra-red (NIR)) and/or combination of frequencies and/or wavelengths. In some embodiments, (e.g., when multiple detectors are used) the received detected composite electronic signals may include and/or be associated with a detector identifier so that a position of receipt for a particular detected composite electronic signal may be known. This location may then be used to analyze the received detected composite electronic signals to determine various factors of the detected light and/or imaged tissue.

In step 1410, a physiological characteristic regarding a pregnant mammal and/or her fetus may be received. Exemplary physiological characteristics include, but are not limited to, fetal depth within the abdomen, fetal position, skin pigmentation of the fetus and/or pregnant mammal, uterine thickness, skin thickness, fetal tissue type, fetal tissue thickness, a density and/or thickness of various layers of tissue (e.g., skin, fat, uterus, subcutaneous fat, amniotic fluid, fetus, etc.) included in the maternal abdomen, and so on. In some embodiments, the physiological characteristic may be determined from analysis of, for example, an image (e.g., ultrasound or MRI) of the maternal abdomen, examples of which are provided in the illustrations of FIGS. 3A and 3B. Additionally, or alternatively, the physiological characteristic may be directly entered by, for example, a physician, user, and/or operator via, for example, measuring the physiological characteristic and/or measuring an aspect of an image of the pregnant mammal's abdomen.

In step 1415, a database, such as database 170, may be queried for information regarding how the physiological characteristic may impact the behavior of light traversing through the pregnant mammal's abdomen and/or her fetus. This determination may be the result of execution of process(es) 500, p 1300, and/or 1100.

In step 1420, the signal received in step 1405 may be analyzed to determine how the physiological characteristic may impact the light's behavior when entering, traveling through, and/or exiting the pregnant mammal's abdomen.

Next, the received detected composite electronic signal may be analyzed to isolate a portion of the signal that corresponds to light that was incident upon the fetus (step 1425). In some instances, this isolated portion of the signal may be referred to herein as a “fetal signal.” Step 1425 may be executed using any appropriate method of isolating the fetal signal from the received detected composite electronic signal. Often times, the fetal signal is associated with the fetal pulsatile signal, which may be used to isolate the fetal signal from the composite electronic signal because the fetal signal (which is optical and included in the optical signal) may correspond, in time, with the fetal pulsatile signal. This correspondence may be used to extract the fetal signal from the composite electronic signal. Appropriate methods include, but are not limited to, reducing noise in the signal via, for example, application of filtering or amplification techniques, determining a portion of the detected composite electronic signal that is contributed by the pregnant mammal and then subtracting or otherwise removing that portion of the detected composite electronic signal from the detected composite electronic signal and/or receiving information regarding fetal heart rate and using that information to lock in (via, for example, a lock-in amplifier) on a portion of the detected composite electronic signal that may be generated and/or influenced by the fetus.

In step 1430, the fetal signal may be analyzed to determine a fetal hemoglobin oxygen saturation level. In some embodiments, two (or more) different detected composite electronic signals (also referred to herein as a first detected composite electronic signal and a second detected composite electronic signal) may be received in step 1405. The first and second detected composite electronic signals may be of two different wavelengths and/or ranges of wavelengths and may be analyzed to create a first fetal signal and a second fetal signal in step 1425. The first and second fetal signals may be analyzed and processed to determine a value of the PPD pulse amplitude at end diastole for each fetal signal thereby determining a first and second PPD pulse amplitude at end diastole, which may be referred to herein as ID1 and ID2, respectively. In some instances, the PPD pulse amplitude at end diastole may be understood and/or referred to as an AC signal or value. Then, the first and second fetal signals may be analyzed and processed to determine a value of the PPD pulse amplitude during systole for each fetal signal thereby determining a first and second PPD pulse amplitude during systole, which may be referred to herein as IS1 and IS2, respectively. In some instances, the PPD pulse amplitude during systole may be understood and/or referred to as an DC signal or value.

Then, a ratio of ratios (also referred to as “R”) may be determined via performing the following calculation via Equation 4a and/or 4b, wherein :

$$R = \frac{{[ ( {I}_{D} - {I}_{S} ) / {I}_{S} ]}_{1}}{{[ ( {I}_{D} - {I}_{S} ) / {I}_{S} ]}_{2}} \qquad Equation 4 a$$

$$R = f ( {I}_{s} , {I}_{D} ) \qquad Equation 4 b$$

In some instances, R may be an average value determined via, a determining a plurality of values for I_{D1}, I_{D2}, I_{S1}, and I_{S2} and then calculating an average value for I_{D1}, I_{D2}, I_{S1}, and I_{S2}, which may be input into Equation(s) 5a, 5b, and/or 5C, discussed below. Additionally, or alternatively, R may be determined by performing the calculation using Equation(s) 4a and/or 4b a plurality of times (e.g., 70, 110, 120, etc.) to determine a plurality of R values that may then be averaged to determine an average R value.

In some instances, the R value determined/calculated via execution of process 1400 may be done on a case-by-case basis for each individual pregnant mammal or fetus to customize, or personalize, the R value for each situation or fetus. In some embodiments, the R value may be relatable to the intrinsic saturation SpO2 values determined from independent control data. This specificity may be of clinical importance because it provides a more accurate determination of R than when a value for R is determined by a pulse oximeter or DOT manufacturer as an average across all situations. In some cases, a R value is provided by a pulse oximeter manufacturer and it is based on an evaluation of experimentally determined results. A problem with this approach is that it presumes conditions under which the pulse oximeter will be used from patient to patient or situation to situation will be relatively uniform as is the case with a finger or ear lobe, which are traditional locations on the body where pulse oximetry measurements are taken. However, such an assumption cannot be made with sufficient certainty in the case of a pregnant mammal and her fetus, which does not exhibit the predictability or uniformity required to have sufficient confidence in a generalized R value determined by a manufacturer under average conditions. At times, determining R may be done a plurality of times during a monitoring session on, for example, a continuous, periodic or as-needed basis to specifically tailor the R value to a point in time or situation. For example, an R value determination may be executed every hour, half-hour, or minute during labor and delivery of the fetus in order to adjust R values when, for example, a fetus and/or the pregnant mammal or her uterus moves and/or muscles expand/contract. Alternatively, an R value may be a measured (independent) quantity that may not be provided by a manufacturer of the oximetry device. A calibration curve associated with these R values may relate to the true SpO2 values is empirically derived and incorporated by the manufacturer.

At times, the determination of the fetal hemoglobin oxygen saturation level may include determining an extinction coefficient for oxygenated hemoglobin (ε0) and deoxygenated hemoglobin (εd) for the first and second fetal signals. The extinction coefficient of hemoglobin may be understood as an absorption coefficient (e.g., μₐ(λ)) of the tissue under study divided by the hemoglobin concentration. The absorption coefficient may be received and/or understood via, for example, execution of process 1100 and/or step 1415. Once the extinction coefficients are determined (via, for example, looking them up in a table and/or a database like database 170), they may be plugged into the following equation (Equation 5a) to determine the fetal hemoglobin oxygen saturation (SpO₂):

$${SpO}_{2} = \frac{{ε}_{d 1} - R ( {1}_{2} / {1}_{1} ) {ε}_{d 2}}{R ( {1}_{2} / {1}_{1} ) ( {ε}_{02} - {ε}_{d 2} ) + ( {ε}_{d 1} - {ε}_{01} )} \qquad Equation 5 a$$

### Where:

ε_{d1}=the extinction coefficient for deoxygenated hemoglobin for λ₁;

ε_{d2}=the extinction coefficient for deoxygenated hemoglobin for λ₂;

ε₀₁=the extinction coefficient for oxygenated hemoglobin for λ₁;

ε₀₂=the extinction coefficient for oxygenated hemoglobin for λ₂;

I₁=the path length for λ₁; and

I₂=the path length for λ₂.

Incorporating one or more of the calibration factors into this calculation may be done via Equations 5b and/or 5c, below , wherein:

SpO₂=g(I₁, I₂, ε₀₁, ε₀₂, P₁, P₂ . . . ) Equation 5b

SpO₂=h(g, C₁, C₂ . . . ) Equation 5c

### Where:

ε₀₁=the extinction coefficient for oxygenated hemoglobin for λ₁;

ε₀₂=the extinction coefficient for oxygenated hemoglobin for λ₂;

I₁=the path length for λ₁;

I₂=the path length for λ₂;

P₁=a calibration factor for a first physiological characteristic;

P₂=a calibration factor for a second physiological characteristic;

g=the generalized equation dependent on the parameters: I₁ I₂, ε_{d1}, ε_{d2}, C₁, C₂, C₃, etc.;

C₁=a first detected electronic signal;

C₂=a second detected electronic signal; and

h=an empirical parametrization equation for relating the physical characteristics and the measured signals Cᵢ

Equation 5b allows for the application of one or more calibration factors for one or more physiological characteristics may be incorporated into the calculation of fetal SpO₂. Equation 5c allows for the application of one or more calibration factors for one or more physiological characteristics to one or more detected electronic signals into a calculation of fetal SpO₂.

Once determined, provision of the fetal oxygen hemoglobin saturation (SpO₂) value to a user (e.g., doctor, nurse, or patient) may be facilitated (step 1435) via, for example, providing the indication to a display device (e.g., display device 155), or a computer (e.g., computer 150) screen or screen of a device (e.g., fetal hemoglobin probe 115).

Optionally, execution of step 1435 may include an indication of whether the fetal hemoglobin oxygen saturation level is pre-ductal or post-ductal to the user in a manner similar to execution of step 1355. The indication of whether the fetal hemoglobin oxygen saturation level is pre-ductal or post-ductal may help a clinician determine whether the fetal hemoglobin oxygen saturation level is low enough to cause concern (e.g., indicate that there is a possibility of fetal acidosis) or to warrant further intervention like a Caesarian section. Further details regarding the execution of step 1340 are provided below with regard to the discussion of process 1400, and, in particular execution of steps 1425 and 1430.

Additionally, or alternatively, execution of step(s) 1425 and/or 1430 may include using one or more impacts of a physiological characteristic on the detected composite electronic signal determined in step 1420 in the form of, for example, adjustment of a scattering coefficient and/or absorption coefficient. For example, the detected composite electronic signals received in step 1405 may be the result of light of two different wavelengths being projected into the maternal abdomen. Exemplary values for the first wavelength (λ1) range between 760 nm and 805 nm and exemplary values for the second wavelength (λ2) range between 808 nm and 830 nm. Often, the light of λ1 and λ2 will be monochromatic or within a narrow band of the electromagnetic spectrum. Light of both wavelengths may be incident upon the pregnant mammal's abdomen and collected via optical cables and passed to one or more detectors like detector 160 and/or may be directly detected by detector 160. The data collected by the detectors may then be interpreted and/or processed via Equation 6, 7a, and 7b below to determine changes in absorption coefficients and changes in oxyhemoglobin saturation (Δ[HbO]) and deoxyhemoglobin saturation (Δ[Hb]), respectively.

$${Δμ}_{a}^{λ} = {ε}_{HbO}^{λ} Δ [ HbO ] + {ε}_{Hb}^{λ} Δ [ HbO ] . \qquad Equation 6$$

$$Δ [ Hb ] = \frac{{ε}_{HbO}^{{λ}_{2}} {Δμ}_{a}^{{λ}_{1}} - {ε}_{HbO}^{{λ}_{1}} {Δμ}_{a}^{{λ}_{2}}}{{ε}_{Hb}^{{λ}_{1}} {ε}_{HbO}^{{λ}^{2}} - {ε}_{Hb}^{{λ}_{2}} {ε}_{HbO}^{{λ}_{1}}} and \qquad Equations ( 7 a ) and ( 7 b )$$

$$Δ [ HbO ] = \frac{{ε}_{HbO}^{{λ}_{2}} {Δμ}_{a}^{{λ}_{1}} - {ε}_{HbO}^{{λ}_{1}} {Δμ}_{a}^{{λ}_{2}}}{{ε}_{Hb}^{{λ}_{1}} {ε}_{HbO}^{{λ}^{2}} - {ε}_{Hb}^{{λ}_{2}} {ε}_{HbO}^{{λ}_{1}}} ,$$

$$( 2 a , b )$$

The value of Δμₐ(λ) may be used in, for example, Equation 6, above, to determine an extinction coefficient for oxygenated hemoglobin (ε0) and/or deoxygenated hemoglobin (εd) for the wavelength λ. These values, for two different wavelengths λ1 and λ2 may then be put into Equations 7a and 7b to determine relative changes in oxyhemoglobin saturation (Δ[HbO]) and deoxyhemoglobin saturation (Δ[Hb]).

A reconstruction algorithm may be applied to account for path length differences between, for example, a source and detector position, a fetal depth, etc. and may be used to reconstruct predicted changes in the absorption coefficient Δμa at a detector. Then, Equations 7a and 7b may be solved to determine changes in oxyhemoglobin saturation (Δ[HbO]) and deoxyhemoglobin saturation (Δ[Hb]) for the fetus. In one exemplary embodiment, these values (Δ[HbO] and A[Hb]) may be used to determine a relative fetal hemoglobin oxygen level. Additionally, or alternatively, values for Δ[HbO] and Δ[Hb] may be used to generate a two- or three-dimensional map of the pregnant mammal's abdomen which show relative changes in oxyhemoglobin saturation (Δ[HbO]) and deoxyhemoglobin saturation (Δ[Hb]). The changes in oxyhemoglobin saturation (Δ[HbO]) and deoxyhemoglobin saturation (Δ[Hb]) may be shown using, for example, grey scale, color-coding and the images may be topographic, cross-sectional, and/or volumetric. While this process does not provide an absolute value for fetal hemoglobin oxygen saturation, it does provide a relative value for fetal hemoglobin oxygen saturation which may be used to monitor fetal hemoglobin oxygen saturation over time to determine changes thereto that may indicate the fetus is in distress as may be the case with a rapidly or slowly declining fetal hemoglobin oxygen saturation level.

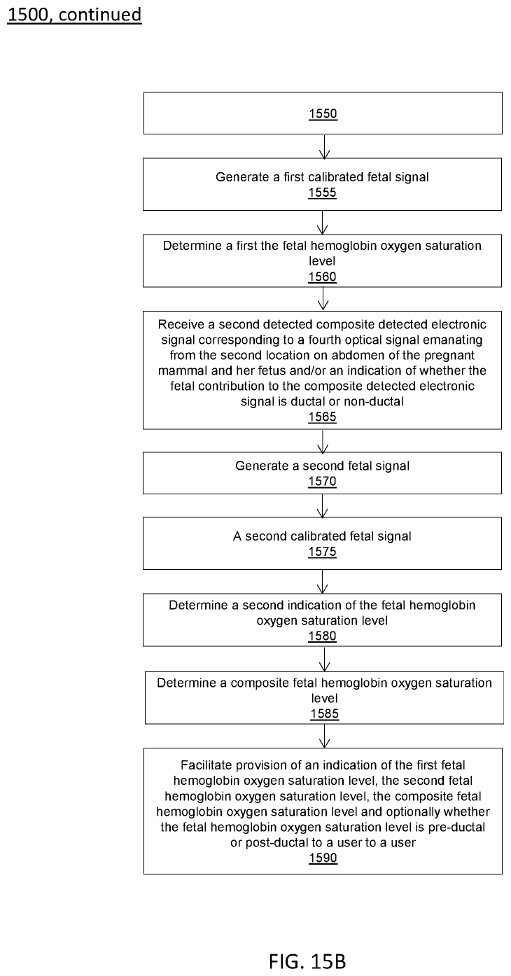

FIG. 15 provides a flowchart illustrating a process 1500 for determining a composite fetal hemoglobin oxygenation saturation level using physiological characteristics of the pregnant mammal and/or fetus. Process 1500 may be performed by, for example, system 100 and/or components thereof. In some embodiments, process 1500 may be executed using a fetal hemoglobin probe like the fetal hemoglobin probe(s) 115 disclosed herein.

In step 1505, a first maternal detected electronic signal may be received by a processor. The first maternal detected electronic signal may be received from a first detector communicatively coupled to the processor. The first maternal detected electronic signal may correspond to a first optical signal emitted from a first location on the pregnant mammal's abdomen that has been detected by a first detector positioned proximate to (on top of) the first location on the pregnant mammal's abdomen and converted into the first maternal detected electronic signal. The first emitted optical signal may be a portion of light projected, by a first light source, into the pregnant mammal's abdomen. In some embodiments, execution of step 1505 may resemble execution of step 905.

In step 1510, the first maternal detected electronic signal may be analyzed to determine optionally determine a physiological characteristic (step 1515). Then, a first calibration factor for the first optical signal emanating from the pregnant mammal at the first location may be determined responsively to the analysis (step 1520). In some embodiments, execution of steps 1510, 1515, and/or 1520 may resemble execution of steps 910, 915, and/or 920, respectively.

In step 1525, a first maternal detected electronic signal may be received by a processor. The first maternal detected electronic signal may be received from a first detector communicatively coupled to the processor. The first maternal detected electronic signal may correspond to a first optical signal emitted from a first location on the pregnant mammal's abdomen that has been detected by a first detector positioned proximate to (on top of) the first location on the pregnant mammal's abdomen and converted into the first maternal detected electronic signal. The first emitted optical signal may be a portion of light projected, by a first light source, into the pregnant mammal's abdomen. In some embodiments, execution of step 1525 may resemble execution of step 1505 but with a different maternal detected electronic signal (i.e., the second maternal detected electronic signal).

In step 1530, the first maternal detected electronic signal may be analyzed to determine optionally determine a physiological characteristic (step 1535). Then, a first calibration factor for the first optical signal emanating from the pregnant mammal at the first location may be determined responsively to the analysis (step 1540). In some embodiments, execution of steps 1510, 1515, and/or 1520 may resemble execution of steps 1510, 1515, and/or 1520, respectively but with a different maternal detected electronic signal (i.e., the second maternal detected electronic signal).

In some embodiments, the first and/or second physiological characteristic(s) and/or the first and/or second calibration factor(s) for the pregnant mammal may be stored in a database. At times, an association between the first physiological characteristic of the pregnant mammal and the first calibration factor and/or an association between the second physiological characteristic of the pregnant mammal and the second calibration factor may be made and this association may be stored in the database.

In step 1545, a first composite detected electronic signal may be received from the first detector. The first detector may be located proximate to the first location on the pregnant mammal's abdomen. In some embodiments like when the pregnant mammal is wearing a fetal hemoglobin probe over a duration of time, the first composite detected electronic signal is received shortly (e.g., 0.5 s, 1 s, 1 minute, etc.) after step 1505 is executed. The first composite detected electronic signal may correspond to a third optical signal emitted from the pregnant mammal's abdomen and a fetus contained therein that has been detected by the first detector and converted into the first composite detected electronic signal. The third emitted optical signal may be a portion of light projected by, for example, the first and/or a third light source into the pregnant mammal's abdomen and onto the fetus contained therein.

The first composite signal may be analyzed or processed using one or more of the processes described herein to isolate a portion of the first composite electronic signal that corresponds to light that was incident upon the fetus thereby generating a first fetal signal (step 1550) using, for example, one or more of the methods disclosed herein. A first calibrated fetal signal may then be generated by applying the first calibration factor to the first fetal signal (step 1555) and a first fetal hemoglobin oxygen saturation level may then be determined (step 1560) using the first calibrated fetal signal. The first fetal hemoglobin oxygen saturation level may be determined using, for example, any of the methods disclosed herein.

In step 1565, a second composite detected electronic signal may be received from the second detector. The second detector may be located proximate to the second location on the pregnant mammal's abdomen. In some embodiments like when the pregnant mammal is wearing a fetal hemoglobin probe over a duration of time, the second composite detected electronic signal is received shortly (e.g., 0.5 s, 1 s, 1 minute, etc.) after step 1505 is executed. The second composite detected electronic signal may correspond to a third optical signal emitted from the pregnant mammal's abdomen and a fetus contained therein that has been detected by the second detector and converted into the second composite detected electronic signal. The third emitted optical signal may be a portion of light projected by, for example, the second and/or a third light source into the pregnant mammal's abdomen and onto the fetus contained therein.

The second composite signal may be analyzed or processed using one or more of the processes described herein to isolate a portion of the second composite electronic signal that corresponds to light that was incident upon the fetus thereby generating a second fetal signal (step 1570) using, for example, one or more of the methods disclosed herein. A second calibrated fetal signal may then be generated by applying the second calibration factor to the second fetal signal (step 1575) and a second fetal hemoglobin oxygen saturation level may then be determined (step 1580) using the second calibrated fetal signal. The second fetal hemoglobin oxygen saturation level may be determined using, for example, any of the methods disclosed herein.

In step 1585, a composite fetal hemoglobin oxygen saturation level may be determined using the first and second fetal hemoglobin oxygen saturation levels. Step 1585 may be executed by, for example, taking an average value of the first and second fetal hemoglobin oxygen saturation level. In some embodiments, process 1500 and/or steps 1545-1580 may be repeated on a continuous, periodic, and/or as-needed basis so that multiple fetal hemoglobin oxygen saturation levels may be determined over time. In these embodiments, the composite fetal hemoglobin oxygen saturation level may include more fetal hemoglobin oxygen saturation levels than the first and second fetal hemoglobin oxygen saturation levels determined in steps 1560 and 1580 and these values may, in some cases, be averaged and/or the composite fetal hemoglobin oxygen saturation level may be a time weighted average of all fetal hemoglobin oxygen saturation levels determined over a given period of time (e.g., 5 minutes, 15 minutes, 1 hour, etc.). In step 1590, an indication of the composite fetal hemoglobin oxygen saturation level may be communicated to a user. In some cases, an indication the first and/or second fetal hemoglobin oxygen saturation level(s) may also be provided in step 1590. Additionally, or alternatively, an indication of whether the fetal blood used to determine the fetal hemoglobin oxygen saturation levels is pre-ductal or post-ductal may also be provided in step 1590.

For the embodiments herein described, the light directed into the pregnant mammal's abdomen and the fetus may be of at least two separate wavelengths and/or frequencies (e.g., red, infrared, near-infrared, etc.) and the received detected electronic signals may correspond to light of these different wavelengths.

Hence, systems, devices, and methods for determining fetal oxygen level have been herein disclosed. In some embodiments, use of the systems, devices, and methods described herein may be particularly useful during the labor and delivery of the fetus (e.g., during the first and/or second stage of labor) because it is difficult to assess fetal health during the labor and delivery process.

In some embodiments, two or more of the processes, or portions thereof, may be combined in any order and executed together.

We claim:

1. A method comprising:
receiving, by a processor, a physiological characteristic of a pregnant mammal;
determining, by the processor, an impact of the physiological characteristic on a behavior of an optical signal projected into the abdomen of the pregnant mammal; and
determining, by the processor, a calibration factor for the optical signal responsively to the impact.

2. The method of claim 1, further comprising:
receiving, by the processor, a composite detected electronic signal from a detector communicatively coupled to the processor, the composite electronic signal corresponding to an optical signal emitted from the pregnant mammal's abdomen and a fetus contained therein that has been detected by the detector and converted into the composite detected electronic signal, the emitted optical signal being a portion of light projected, by a light source, into the pregnant mammal's abdomen and onto the fetus contained therein;
generating, by the processor, a fetal signal by isolating a portion of the composite detected electronic signal that corresponds to light that was incident upon the fetus;
generating, by the processor, a calibrated fetal signal by applying the calibration factor to the fetal signal;
determining, by the processor, a fetal hemoglobin oxygen saturation level using the calibrated fetal signal; and
facilitating, by the processor, provision of the fetal hemoglobin oxygen saturation level to a user.

3. The method of claim 1 or 2, wherein determining the calibration factor for the optical signal responsively to the impact comprises:
querying, by the processor, a database for a calibration factor that
corresponds to the physiological characteristic.

4. The method of claim 1, 2, or 3, further comprising:
receiving, by the processor, an indication of whether the fetal signal corresponds to pre-ductal or post-ductal blood; and
providing, by the processor, the indication of whether the fetal signal corresponds to pre-ductal or post-ductal blood when facilitating provision of the fetal hemoglobin oxygen saturation level to the user.

5. The method of any of claims 1-4, further comprising:
receiving, by the processor, a maternal detected electronic signal from a detector communicatively coupled to the processor, the maternal detected electronic signal corresponding to an optical signal emitted from the pregnant mammal's abdomen that has been detected by the detector and converted into the maternal detected electronic signal, the emitted optical signal being a portion of light projected, by a light source, into the pregnant mammal's abdomen;
analyzing, by the processor, the maternal detected electronic signal, wherein the physiological characteristic of the pregnant mammal is determined responsively to the analysis.

6. The method of claims 5, further comprising:
storing, by the processor, the determined physiological characteristic and the calibration factor for the pregnant mammal in a database.

7. The method of any of claims 1-6, wherein the physiological characteristic is received from at least one of an ultra-sound device, a Doppler device, an image of the pregnant mammal's abdomen, a Fitzpatrick scale reading, manually-operated calipers, a blood measurement device, an oximeter, a pulse oximeter, a scale.

8. The method of any of claims 1-7, wherein the physiological characteristic is intrinsic.

9. The method of any of claims 1-8, wherein the physiological characteristic is extrinsic.

10. The method of any of claims 1-9, wherein the physiological characteristic is the pregnant mammal's age, the pregnant mammal's weight, and the pregnant mammal's body mass index.

11. The method of any of claims 1-10, wherein the received physiological characteristic is a skin color of the pregnant mammal, further wherein the determination of the impact of the physiological characteristic on the behavior of the optical signal includes determining how much of the optical signal is absorbed by the pregnant mammal's skin color.

12. The method of any of claims 1-10, wherein the received physiological characteristic is a thickness of a muscle layer in the pregnant mammal's abdomen, further wherein the determination of the impact of the physiological characteristic on the behavior of the optical signal includes determining how much of the optical signal is absorbed by the muscle layer in the pregnant mammal's abdomen.

13. The method of any of claims 1-10, wherein the received physiological characteristic is a thickness of an adipose layer in the pregnant mammal's abdomen, further wherein the determination of the impact of the physiological characteristic on the behavior of the optical signal includes determining how much of the optical signal is scattered by the adipose layer in the pregnant mammal's abdomen.

14. The method of any of claims 1-10, wherein the received physiological characteristic is a body mass index for the pregnant mammal, further wherein the determination of the impact of the physiological characteristic on the behavior of the optical signal includes determining how much of the optical signal is scattered or absorbed by the pregnant mammal's abdomen.

15. The method of any of claims 1-10, wherein the received physiological characteristic is a thickness of the pregnant mammal's abdomen, further wherein the determination of the impact of the physiological characteristic on the behavior of the optical signal includes determining how much of the optical signal is absorbed by the pregnant mammal's abdomen.

16. The method of any of claims 1-10, wherein the received physiological characteristic is a thickness of the pregnant mammal's abdomen, further wherein the determination of the impact of the physiological characteristic on the behavior of the optical signal includes determining how much of the optical signal is scattered by the pregnant mammal's abdomen.

17. The method of any of claims 1-10, wherein the received physiological characteristic is a hemoglobin concentration of the pregnant mammal's blood, further wherein the determination of the impact of the physiological characteristic on the behavior of the optical signal includes determining how much of the optical signal is absorbed by the pregnant mammal's hemoglobin.

18. The method of any of claims 1-10, wherein the received physiological characteristic is a hemoglobin oxygen saturation of the pregnant mammal's blood, further wherein the determination of the impact of the physiological characteristic on the behavior of the optical signal includes determining how much of the optical signal is absorbed by the pregnant mammal's oxygenated and deoxygenated hemoglobin.

19. A method comprising:
receiving, by a processor, a maternal detected electronic signal from a detector communicatively coupled to the processor, the maternal detected electronic signal corresponding to an optical signal emitted from the pregnant mammal's abdomen that has been detected by the detector and converted into the maternal detected electronic signal, the emitted optical signal being a portion of light projected, by a light source, into the pregnant mammal's abdomen; and
analyzing, by the processor, the maternal detected electronic signal, to determine a physiological characteristic of the pregnant mammal; and
determining, by the processor, a calibration factor for the optical signal emanating from the pregnant mammal responsively to the analysis.

20. The method of claim 19, further comprising:
associating, by the processor, the physiological characteristic of the pregnant mammal with the calibration factor;
storing, by the processor, the association between the physiological characteristic of the pregnant mammal to the calibration factor in a database.

21. The method of any of claim 19 or 20, further comprising:
receiving, by the processor, a composite detected electronic signal from a detector communicatively coupled to the processor, the composite detected electronic signal corresponding to an optical signal emitted from the pregnant mammal's abdomen and a fetus contained therein that has been detected by the detector and converted into the composite detected electronic signal, the emitted optical signal being a portion of light projected, by a light source, into the pregnant mammal's abdomen and onto the fetus contained therein;
generating, by the processor, a fetal signal by isolating a portion of the composite detected electronic signal that corresponds to light that was incident upon the fetus;
generating, by the processor, a calibrated fetal signal by applying the calibration factor to the fetal signal;
determining, by the processor, a fetal hemoglobin oxygen saturation level using the calibrated fetal signal; and
facilitating, by the processor, provision of the fetal hemoglobin oxygen saturation level to a user.

22. The method of any of claims 19-22, wherein determining the calibration factor for
the optical signal responsively to the impact comprises:
querying, by the processor, a database for a calibration factor that
corresponds to the physiological characteristic.

23. The method of any of claims 19-23, further comprising:
receiving, by the processor, an indication of whether the fetal signal corresponds to pre-ductal or post-ductal blood; and
providing, by the processor, the indication of whether the fetal signal corresponds to pre-ductal or post-ductal blood when facilitating provision of the fetal hemoglobin oxygen saturation level to the user.

24. The method of any of claims 19-23, wherein the physiological characteristic is intrinsic.

25. The method of any of claims 19-23, wherein the physiological characteristic is extrinsic.

26. The method of any of claims 19-25, wherein the determined physiological characteristic is a skin color of the pregnant mammal and the calibration factor pertains to how much of the optical signal is absorbed by the pregnant mammal's skin color.

27. The method of any of claims 19-25, wherein the determined physiological characteristic is a thickness of a muscle layer in the pregnant mammal's abdomen and the calibration factor pertains to how much of the optical signal is absorbed by the muscle layer in the pregnant mammal's abdomen.

28. The method of any of claims 19-25, wherein the determined physiological characteristic is a thickness of an adipose layer in the pregnant mammal's abdomen and the calibration factor pertains to how much of the optical signal is scattered by the adipose layer in the pregnant mammal's abdomen.

29. The method of any of claims 19-25, wherein the determined physiological characteristic is a thickness of the pregnant mammal's abdomen and the calibration factor pertains to how much of the optical signal is absorbed by the pregnant mammal's by the pregnant mammal's abdomen.

30. The method of any of claims 19-25, wherein the determined physiological characteristic is a thickness of the pregnant mammal's abdomen and the calibration factor pertains to how much of the optical signal is scattered by the pregnant mammal's abdomen.

31. The method of any of claims 19-25, wherein the determined physiological characteristic is a hemoglobin concentration of the pregnant mammal's blood and the calibration factor pertains to how much of the optical signal is absorbed by the pregnant mammal's hemoglobin.

32. The method of any of claims 19-25, wherein the determined physiological characteristic is a hemoglobin oxygen saturation of the pregnant mammal's blood and the calibration factor pertains to how much of the optical signal is absorbed by the pregnant mammal's oxygenated and deoxygenated hemoglobin.

33. A method comprising:
receiving, by the processor, a first maternal detected electronic signal from a first detector communicatively coupled to the processor, the first maternal detected electronic signal corresponding to a first optical signal emitted from a first location on the pregnant mammal's abdomen that has been detected by a first detector positioned proximate to the first location of the pregnant mammal's abdomen and converted into the first maternal detected electronic signal, the first emitted optical signal being a portion of light projected, by a first light source, into the pregnant mammal's abdomen;
analyzing, by the processor, the first maternal detected electronic signal, to determine a first calibration factor for the first optical signal emanating from the pregnant mammal at the first location responsively to the analysis;
receiving, by the processor, a second maternal detected electronic signal from a second detector communicatively coupled to the processor, the second maternal detected electronic signal corresponding to a second optical signal emitted from the pregnant mammal's abdomen that has been detected by a second detector positioned in a second location on the pregnant mammal's abdomen and converted into the second maternal detected electronic signal, the second emitted optical signal being a portion of light projected, by a second light source, into the pregnant mammal's abdomen;
analyzing, by the processor, the second maternal detected electronic signal, to determine a second calibration factor for the second optical signal emanating from the pregnant mammal at the second location responsively to the analysis.

34. The method of claim 33, further comprising:
analyzing, by the processor, the first maternal detected electronic signal, to determine a first physiological characteristic for the first location on the pregnant mammal's abdomen responsively to the analysis.

35. The method of claim 34, further comprising:
storing, by the processor, the first physiological characteristic and the first calibration factor for the pregnant mammal in a database.

36. The method of claim 34 or 35, further comprising:
associating, by the processor, the first physiological characteristic of the pregnant mammal with the first calibration factor;
storing, by the processor, the association between the first physiological characteristic of the pregnant mammal to the first calibration factor.

37. The method of any of claims 33-36, further comprising:
analyzing, by the processor, the second maternal detected electronic signal, to determine a second physiological characteristic for the second location on the pregnant mammal's abdomen responsively to the analysis.

38. The method of any of claims 37, further comprising:
storing, by the processor, the second physiological characteristic and the calibration factor for the pregnant mammal in a database.

39. The method of claim 38, further comprising:
associating, by the processor, the second physiological characteristic of the pregnant mammal with the second calibration factor;
storing, by the processor, the association between the second physiological characteristic of the pregnant mammal to the second calibration factor.

40. The method of any of claims 33-38, further comprising:
receiving, by the processor, a first composite detected electronic signal from the first detector the first composite detected electronic signal corresponding to a third optical signal emitted from the pregnant mammal's abdomen and a fetus contained therein that has been detected by the first detector and converted into the first composite detected electronic signal, the third emitted optical signal being a portion of light projected, by the first light source, into the pregnant mammal's abdomen and onto the fetus contained therein;
generating, by the processor, a first fetal signal by isolating a portion of the first composite electronic signal that corresponds to light that was incident upon the fetus;
generating, by the processor, a first calibrated fetal signal by applying the first calibration factor to the first fetal signal;
determining, by the processor, a first fetal hemoglobin oxygen saturation level using the first calibrated fetal signal; and
facilitating, by the processor, provision of the fetal hemoglobin oxygen saturation level to a user.

41. The method of any of claims 33-39, further comprising:
receiving, by the processor, a second composite detected electronic signal from the second detector, the second composite detected electronic signal corresponding to a fourth optical signal emitted from the pregnant mammal's abdomen and a fetus contained therein that has been detected by the second detector and converted into the second composite detected electronic signal, the emitted fourth optical signal being a portion of light projected, by the second light source, into the pregnant mammal's abdomen and onto the fetus contained therein;
generating, by the processor, a second fetal signal by isolating a portion of the second composite electronic signal that corresponds to light that was incident upon the fetus;
generating, by the processor, a second calibrated fetal signal by applying the second calibration factor to the first fetal signal;
determining, by the processor, a second fetal hemoglobin oxygen saturation level using the first calibrated fetal signal;
determining, by the processor, a composite fetal hemoglobin oxygen saturation level using the first and second fetal hemoglobin oxygen saturation levels; and
facilitating, by the processor, provision of the composite fetal hemoglobin oxygen saturation level to a user.
